(19) United States
(12) Patent Application Publication
(10) Pub. No.: US 2024/0369924 A1
(43) Pub. Date: Nov. 7, 2024

(54) RESIST COMPOSITION AND RESIST FILM FORMING METHOD USING SAME

(71) Applicant: MITSUBISHI GAS CHEMICAL COMPANY, INC., Tokyo (JP)

(72) Inventors: Takumi OKADA, Niigata (JP); Ryosuke HOSHINO, Niigata (JP); Hideyuki SATO, Niigata (JP); Masayuki KATAGIRI, Niigata (JP); Shu SUZUKI, Kanagawa (JP); Masatoshi ECHIGO, Tokyo (JP)

(73) Assignee: MITSUBISHI GAS CHEMICAL COMPANY, INC., Tokyo (JP)

(21) Appl. No.: 18/291,740

(22) PCT Filed: Jul. 25, 2022
(86) PCT No.: PCT/JP2022/028576

§ 371 Date: Jan. 24, 2024

(30) Foreign Application Priority Data
Jul. 30, 2021 (JP) 2021-125370
Sep. 10, 2021 (JP) 2021-147668

(51) Int. Cl. G03F 7/004; C08F 220/18; C08F 220/28; G03F 7/039

(52) U.S. Cl. CPC G03F 7/0048; C08F 220/1812; C08F 220/283; G03F 7/039

(57) ABSTRACT

A resist composition includes: (A) a resin; and (B) a solvent containing: (B1) a compound represented by the following general formula (b-1), wherein the content of the active component is 45% by mass or less based on the total amount of the resist composition:
wherein R¹ is an alkyl group having 1 to 10 carbon atoms.

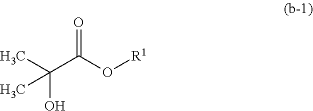

### TECHNICAL FIELD

The present invention relates to a resist composition, and a method for forming a resist film using the resist composition.

### BACKGROUND ART

In the manufacture of semiconductor devices and liquid crystal devices, fine processing is performed by lithography involving use of photoresist materials. In particular, in the manufacture of semiconductor devices, further miniaturization of pattern dimensions has been required along with increases in the integration and speed of LSI, in recent years. To correspond to such miniaturization of pattern dimensions, the light source for lithography used upon forming resist patterns has been shifted from KrF excimer laser (248 nm) to ArF excimer laser (193 nm), which has a shorter wavelength.

For example, Patent Literature 1 discloses an invention relating to a positive type resist composition including a resin in which the hydroxyl group in the carboxy group of a (meth)acrylic acid is protected by an acid-dissociable dissolution inhibiting group, as a photoresist material adaptable to the resist pattern formation using ArF excimer laser.

In recent years, the development of a three-dimensional structure device has been advanced, in which the capacity of a memory is increased by lamination (stacking cells) in addition to the miniaturization of pattern dimensions. Then, in the manufacture of the three-dimensional structure device, a resist pattern is formed after forming a thick resist film having a thicker film thickness than that of conventional films.

### CITATION LIST

### Patent Literature

Patent Literature 1: Japanese Patent Laid-Open No. 2003-241385

### SUMMARY OF INVENTION

### Technical Problem

As described above, characteristics required of the photoresist material used in the manufacture of various devices such as semiconductor devices and liquid crystal devices are different depending on the type of devices. Therefore, a photoresist material capable of forming a resist film suitable for the manufacture of various devices is required.

### Solution to Problem

The present invention provides a resist composition which contains a resin and a solvent including a compound having a specific structure and in which the content of the active ingredient is limited to a predetermined value or less, and also provides a method for forming a resist film using the resist composition.

Specifically, the present invention provides the following [1] to [14].
[1] A resist composition comprising:
   (A) a resin, and
   (B) a solvent comprising: (B1) a compound represented by the following general formula (b-1), wherein
   a content of an active component is 45% by mass or less based on the total amount of the resist composition:

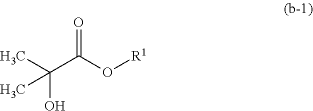

wherein R¹ is an alkyl group having 1 to 10 carbon atoms.
[2] The resist composition according to the above [1], further comprising: (C) at least one additive selected from the group consisting of a photosensitizer and an acid generating agent.
[3] The resist composition according to the above [1] or [2], wherein R¹ in the general formula (b-1) is a methyl group, an ethyl group, an n-propyl group, an i-propyl group, an n-butyl group, an i-butyl group, an s-butyl group, or a t-butyl group.
[4] The resist composition according to any one of the above [1] to [3], wherein R¹ in the general formula (b-1) is an ethyl group, an n-propyl group, an i-propyl group, an n-butyl group, an i-butyl group, an s-butyl group, or a t-butyl group.
[5] The resist composition according to any one of the above [1] to [4], wherein the solvent (B) comprises: (B2) a solvent other than the compound (B1).
[6] The resist composition according to the above [5], wherein the solvent (B) comprises one or more selected from the group consisting of methyl α-methoxyisobutyrate, methyl α-formyloxyisobutyrate, methyl «-acetyloxyisobutyrate, and methyl 3-hydroxyisobutyrate as the solvent (B2).
[7] The resist composition according to the above [5], wherein the solvent (B) comprises one or more selected from the group consisting of methyl α-methoxyisobutyrate, methyl α-formyloxyisobutyrate, methyl α-acetyloxyisobutyrate, methyl 3-hydroxyisobutyrate, and 1-methoxy-2-propanol as the solvent (B2).
[8] The resist composition according to any one of the above [5] to [7], wherein the solvent (B2) is contained in an amount of 100% by mass or less based on the total amount (100% by mass) of the compound (B1).
[9] The resist composition according to the above [8], wherein the solvent (B2) is contained in an amount of less than 70% by mass based on the total amount (100% by mass) of the compound (B1).
[10] The resist composition according to the above [8] or [9], wherein the solvent (B2) is contained in an amount of 0.0001% by mass or more based on the total amount (100% by mass) of the compound (B1).
[11] The resist composition according to any one of the above [5] to [10], wherein the solvent (B2) is contained in an amount less than 100% by mass based on the total amount (100% by mass) of the resist composition.
[12] The resist composition according to any one of the above [1] to [11], wherein the resin (A) comprises a novolac resin (A1).
[13] The resist composition according to any one of the above [1] to [11], wherein the resin (A) comprises: (A2) a resin having at least one of (a2-1) a constitutional unit derived from a phenolic hydroxyl group-containing compound, and (a2-2) a constitutional unit capable of forming an acid functional group by being decomposed by an action of an acid, a base, or heat.
[14] The resist composition according to any one of the above [1] to [11], wherein the resin (A) comprises: (A3) a resin having (a3-1) a constitutional unit having an adamantane structure.
[15] The resist composition according to the above [14], wherein the resin (A3) is a copolymer having (a3-2) a constitutional unit having a lactone structure together with the constitutional unit (a3-1).
[16] The resist composition according to the above or [15], wherein a content of (a3-1α) a constitutional unit having an adamantane structure substituted with a hydroxy group is less than 50 mol %, based on the total amount of the constitutional unit of the resin (A3).
[17] The resist composition according to any one of the above [1] to [11], wherein the resin (A) comprises: (A4) a resin having any two or more constitutional units of (a2-1) the constitutional unit derived from a phenolic hydroxyl group-containing compound, (a2-2) the constitutional unit capable of being decomposed by an action of an acid, a base, or heat to form an acid functional group, (a3-1) the constitutional unit having an adamantane structure, and (a3-2) the constitutional unit having a lactone structure.
[18] A method for forming a resist film comprising:
   step (1): a step of applying the resist composition according to any one of the above [1] to [17] on a substrate to form a coating film,
   step (2): a step of performing heat treatment after step (1), and
   step (3): a step of forming a resist pattern.

### Advantageous Effects of Invention

The resist composition of a suitable aspect of the present invention can form a resist film suitable for the manufacture of various devices though the content of the active component including the resin is limited to a predetermined value or less.

### DESCRIPTION OF EMBODIMENTS

### [Resist Composition]

The resist composition of the present invention contains: (A) a resin (hereinafter, also referred to as the “component (A)”); and (B) a solvent containing: (B1) a compound represented by the general formula (b-1) (hereinafter, also referred to as the “component (B)”). Although the resist composition of the present invention is used to form a resist film, the “resist film” does not include films used for the underlayer of a resist (e.g., resist auxiliary films such as a resist intermediate layer film and a resist underlayer film).

The resist composition of one aspect of the present invention preferably further contains: (C) at least one additive selected from the group consisting of a photosensitizer and an acid generating agent (hereinafter, also referred to as the “component (C)”).

Then, in the resist composition of the present invention, the content of the active component is limited to 45% by mass or less, based on the total amount (100% by mass) of the resist composition.

As used herein, the “active component” refers to the components excluding the component (B) among the components contained in the resist composition. Specifically, the active component encompasses the resin (A) and the additive (C), as well as an acid cross-linking agent, an acid diffusion controlling agent, a dissolution accelerator, a dissolution controlling agent, a sensitizing agent, a surfactant, an organic carboxylic acid or phosphorus oxoacid or a derivative thereof, a dye, a pigment, an adhesion aid, a halation preventing agent, a storage stabilizing agent, a defoaming agent, a shape improver, and the others that may be contained as other additives as described below.

Typically, for example, a thick resist film is required to be formed to manufacture a three-dimensional structure device. However, when a resist composition having a low resin content is used, it is difficult to form a thick resist film.

In contrast, the resist composition of the present invention can be a photoresist material capable of forming a thick resist film owing to use of the compound represented by the general formula (b-1) as the solvent, in spite of a reduced content of the active component including the resin of 45% by mass or less. In addition, since the content of the active component is reduced to 45% by mass or less, the resist composition of the present invention has an economical advantage.

In the resist composition of one aspect of the present invention, the content of the active component may be appropriately set depending on the application, and may be 42% by mass or less, 40% by mass or less, 36% by mass or less, 31% by mass or less, 26% by mass or less, 23% by mass or less, 20% by mass or less, 18% by mass or less, 16% by mass or less, 12% by mass or less, 10% by mass or less, 6% by mass or less, or 3% by mass or less, based on the total amount (100% by mass) of the resist composition.

On the other hand, the lower limit of the content of the active component is appropriately set depending on the application, and the content may be 1% by mass or more, 2% by mass or more, 4% by mass or more, 7% by mass or more, or 10% by mass or more, based on the total amount (100% by mass) of the resist composition.

The range of the content of the active component can be specified by any combination of an upper limit value and a lower limit value appropriately selected from the options each mentioned above.

In the resist composition of one aspect of the present invention, the content of the component (A) in the active component is preferably 50 to 100% by mass, more preferably 60 to 100% by mass, further preferably 70 to 100% by mass, further more preferably 75 to 100% by mass, and particularly preferably 80 to 100% by mass, based on the total amount (100% by mass) of the active component contained in the resist composition, in view of producing a photoresist material capable of forming a thick resist film.

The resist composition of one aspect of the present invention may contain other components in addition to the above components (A) to (C) depending on the application.

However, in the resist composition of one aspect of the present invention, the total content of the components (A), (B), and (C) is preferably 30 to 100% by mass, more preferably 40 to 100% by mass, further preferably 60 to 100% by mass, further more preferably 80 to 100% by mass, and particularly preferably 90 to 100% by mass, based on the total amount (100% by mass) of the resist composition.

Hereinafter, details of each component contained in the resist composition of one aspect of the present invention will be described.

### <Component (A): Resin>

The resin (A) contained in the resist composition of one aspect of the present invention is not particularly limited. A known resin for photoresists for known g-line, i-line, KrF excimer laser, ArF excimer laser, EUV, or EB can be used, and an appropriate resin is selected depending on the application. As used herein, the “resin” encompasses a polymer having a predetermined constitutional unit, and also a compound having a predetermined structure.

The weight average molecular weight (Mw) of the resin used in one aspect of the present invention is preferably 400 to 50,000, more preferably 1,000 to 40,000, and further preferably 1,000 to 30,000.

In the resist composition of the present invention, the content of the component (A) may be appropriately set depending on the application, and may be 45% by mass or less, 42% by mass or less, 40% by mass or less, 35% by mass or less, 31% by mass or less, 26% by mass or less, 23% by mass or less, 20% by mass or less, 18% by mass or less, 16% by mass or less, 12% by mass or less, 10% by mass or less, 6% by mass or less, or 3% by mass or less, based on the total amount (100% by mass) of the resist composition.

The lower limit of the content of the component (A) is also appropriately set depending on the application, and the content may be 1% by mass or more, 2% by mass or more, 4% by mass or more, 7% by mass or more, or 10% by mass or more, based on the total amount (100% by mass) of the resist composition.

The range of the content of the component (A) can be specified by any combination of an upper limit value and a lower limit value appropriately selected from the options each mentioned above.

For example, in the case of producing a photoresist material for exposure to ultraviolet rays such as g-line and i-line to manufacture a liquid crystal device, the resin (A) preferably contains a novolac resin (A1).

In the case of producing a photoresist material for KrF excimer laser, the resin (A) preferably contains: (A2) a resin having at least one of a constitutional unit derived from a phenolic hydroxyl group-containing compound and a constitutional unit capable of being decomposed by an action of an acid, a base, or heat to form an acid functional group.

In the case of producing a photoresist material for ArF excimer laser, the resin (A) preferably contains: (A3) a resin having a constitutional unit having an adamantane structure.

In the case of producing a photoresist material for EUV, the resin (A) preferably contains: (A4) a resin having any two or more constitutional units of a constitutional unit derived from a phenolic hydroxyl group-containing compound, a constitutional unit capable of being decomposed by an action of an acid, a base, or heat, a constitutional unit having an adamantane structure to form an acid functional group, and a constitutional unit having a lactone structure (provided that, the resin (A2) and the resin (A3) are excluded).

The resin (A) contained in the resist composition of one aspect of the present invention may contain only one selected from the group consisting of these resins (A1), (A2), (A3), and (A4), or may contain two or more thereof in combination.

As the resin (A), a resin other than the resins (A1), (A2), (A3), or (A4) may be contained.

However, the total content of the resins (A1), (A2), (A3), and (A4) in the resin (A) used in one aspect of the present invention is preferably 60 to 100% by mass, more preferably 70 to 100% by mass, further preferably 80 to 100% by mass, further more preferably 90 to 100% by mass, and particularly preferably 95 to 100% by mass, based on the total amount (100% by mass) of the resin (A).

Hereinafter, these resins (A1), (A2), (A3), and (A4) will be described.

### [Novolac Resin (A1)]

Examples of the novolac resin (A1) used in one aspect of the present invention include resins obtained by reacting a phenol with at least one of an aldehyde and a ketone in the presence of an acid catalyst (e.g., hydrochloric acid, sulfuric acid, and oxalic acid). The novolac resin (A1) is not particularly limited, and a known resin is used. For example, resins exemplified in Japanese Patent Laid-Open No. 2009-173623, International Publication No. WO 2013-024778, and International Publication No. WO 2015-137485 can be used.

Examples of the phenol include phenol, orthocresol, metacresol, paracresol, 2,3-dimethylphenol, 2,5-dimethylphenol, 3,4-dimethylphenol, 3,5-dimethylphenol, 2,4-dimethylphenol, 2,6-dimethylphenol, 2,3,5-trimethylphenol, 2,3,6-trimethylphenol, 2-t-butylphenol, 3-t-butylphenol, 4-t-butylphenol, 2-methylresorcinol, 4-methylresorcinol, 5-methylresorcinol, 4-t-butylcatechol, 2-methoxyphenol, 3-methoxyphenol, 2-propylphenol, 3-propylphenol, 4-propylphenol, 2-isopropylphenol, 2-methoxy-5-methylphenol, 2-t-butyl-5-methylphenol, thymol, isothymol, 4,4′-biphenol, 1-naphthol, 2-naphthol, hydroxyanthracene, hydroxypyrene, 2,6-dihydroxynaphthalene, and 2,6-dihydroxynaphthalene.

These phenols may be used singly or in combination of two or more thereof.

Examples of the aldehyde include formaldehyde, paraformaldehyde, trioxane, acetaldehyde, propionaldehyde, benzaldehyde, phenylacetaldehyde, α-phenylpropionaldehyde, β-phenylpropionaldehyde, benzaldehyde, 4-biphenylaldehyde, o-hydroxybenzaldehyde, m-hydroxybenzaldehyde, p-hydroxybenzaldehyde, o-chlorobenzaldehyde, m-chlorobenzaldehyde, p-chlorobenzaldehyde, o-methylbenzaldehyde, m-methylbenzaldehyde, p-methylbenzaldehyde, p-ethylbenzaldehyde, 3,4-dimethylbenzaldehyde, p-n-propylbenzaldehyde, p-n-butylbenzaldehyde, terephthalaldehyde, 1-naphthaldehyde, and 2-naphthaldehyde.

Examples of the ketone include acetone, methyl ethyl ketone, diethyl ketone, acetophenone, and diphenyl ketone.

These aldehydes and ketones may be used singly or in combination of two or more thereof.

Among these, the novolac resin (A1) used in one aspect of the present invention is preferably a resin obtained by a condensation reaction of cresol with an aldehyde, more preferably a resin obtained by a condensation reaction of at least one of metacresol and paracresol with at least one of formaldehyde and paraformaldehyde, and further preferably a resin obtained by a condensation reaction of combination of metacresol and paracresol with at least one of formaldehyde and paraformaldehyde.

When metacresol and paracresol are used in combination, the blending ratio by mass of metacresol to paracresol [metacresol/paracresol] as starting materials is preferably 10/90 to 90/10, more preferably 20/80 to 80/20, and further preferably 50/50 to 70/30.

As the novolac resin (A1) used in one aspect of the present invention, commercial products such as “EP4080G” and “EP4050G” (both manufactured by ASAHI YUKIZAI CORPORATION, cresol novolac resin) may be used.

The weight average molecular weight (Mw) of the novolac resin (A1) used in one aspect of the present invention is preferably 500 to 30,000, more preferably 1,000 to 20,000, further preferably 1,000 to 15,000, and further more preferably 1,000 to 10,000.

### [Resin (A2)]

The resin (A2) used in one aspect of the present invention is not particularly limited, and a known resin is used. However, the resin (A2) is desirably a resin having at least one of (a2-1) a constitutional unit derived from a phenolic hydroxyl group-containing compound, and (a2-2) a constitutional unit capable of being decomposed by an action of an acid, a base, or heat to form an acid functional group. The resin (A2) is more preferably a copolymer having both the constitutional unit (a2-1) and the constitutional unit (a2-2).

The solubility in the alkaline developer can be increased when the resin (A2) is the resin having at least one of the constitutional unit (a2-1) and the constitutional unit (a2-2).

In the resin (A2) used in one aspect of the present invention, the total content of the constitutional unit (a2-1) and the constitutional unit (a2-2) is preferably 30 mol % or more, more preferably 50 mol % or more, further preferably 60 mol % or more, further more preferably 70 mol % or more, and particularly preferably 80 mol % or more, based on the total amount (100 mol %) of the constitutional unit of the resin (A2).

When the resin (A2) used in one aspect of the present invention is the copolymer having both the constitutional unit (a2-1) and the constitutional unit (a2-2), the content ratio of the constitutional unit (a2-1) to the constitutional unit (a2-2) [(a2-1)/(a2-2)] is preferably 1/10 to 10/1, more preferably 1/5 to 8/1, further preferably 1/2 to 6/1, and further more preferably 1/1 to 4/1 in a molar ratio.

Examples of the phenolic hydroxyl group-containing compound for the constitutional unit (a2-1) include hydroxystyrene (o-hydroxystyrene, m-hydroxystyrene, and p-hydroxystyrene) and isopropenylphenol (o-isopropenylphenol, m-isopropenylphenol, and p-isopropenylphenol), and hydroxystyrene is preferable.

Examples of the acid functional group that may be formed from the constitutional unit (a2-2) when the unit is decomposed by the action of an acid, a base, or heat include a phenolic hydroxyl group and a carboxyl group.

Examples of the monomer for the constitutional unit capable of forming a phenolic hydroxyl group include hydroxy(α-methyl)styrenes protected by an acetal group, such as p-(1-methoxyethoxy)styrene, p-(1-ethoxyethoxy)styrene, p-(1-n-propoxyethoxy) styrene, p-(1-i-propoxyethoxy)styrene, p-(1-cyclohexyloxyethoxy)styrene, and α-methyl substituents thereof; p-acetoxystyrene, t-butoxycarbonylstyrene, t-butoxystyrene, and α-methyl substituents thereof.

These may be used singly or in combination of two or more thereof.

Examples of the monomer for the constitutional unit capable of forming a carboxyl group include (meth)acrylates protected by an acid decomposable ester group such as t-butyl (meth)acrylate, tetrahydropyranyl (meth)acrylate, 2-methoxybutyl (meth)acrylate, 2-ethoxyethyl (meth)acrylate, 2-t-butoxycarbonylethyl (meth)acrylate, 2-benzyloxycarbonylethyl (meth)acrylate, 2-phenoxycarbonylethyl (meth)acrylate, 2-cyclohexyloxycarbonyl (meth)acrylate, 2-isobornyloxycarbonylethyl (meth)acrylate, and 2-tricyclodecarbonyloxycarbonylethyl (meth)acrylate.

These may be used singly or in combination of two or more thereof.

Among these, as the monomer for the constitutional unit (a2-2), at least one selected from the group consisting of t-butyl (meth)acrylate, tetrahydropyranyl (meth)acrylate, 2-cyclohexyloxycarbonylethyl (meth)acrylate, and p-(1-ethoxyethoxy)styrene is preferable.

The resin (A2) used in one aspect of the present invention, which is a resin having at least one of the constitutional unit (a2-1) and the constitutional unit (a2-2) as described above, may also have other constitutional units different from these constitutional units.

Examples of the monomer for such other constitutional units include alkyl (meth)acrylates; hydroxy group-containing monomers; epoxy group-containing monomers; cycloaliphatic structure-containing monomers; olefins such as ethylene, propylene, and isobutylene; halogenated olefins such as vinyl chloride and vinylidene chloride; diene monomers such as butadiene, isoprene, and chloroprene; aromatic vinyl monomers such as styrene, α-methylstyrene, p-methylstyrene, p-chlorostyrene, and p-methoxystyrene; cyano group-containing vinyl monomers such as (meth)acrylonitrile and cyanated vinylidene; (meth)acrylamides such as (meth)acrylamide, N,N-dimethyl (meth)acrylamide, N,N-dimethylol (meth)acrylamide; and heteroatom-containing cycloaliphatic vinyl monomers such as (meth)acryloylmorpholine, N-vinyl pyrrolidone, and N-vinyl caprolactam.

Examples of the alkyl (meth)acrylate include compounds other than the monomer for the constitutional unit (a2-2), such as methyl (meth)acrylate, ethyl (meth)acrylate, and propyl (meth)acrylate (n-propyl (meth)acrylate and i-propyl (meth)acrylate).

Examples of the hydroxy group-containing monomer include hydroxyalkyl (meth)acrylates such as 2-hydroxyethyl (meth)acrylate, 2-hydroxypropyl (meth)acrylate, 3-hydroxypropyl (meth)acrylate, 2-hydroxybutyl (meth)acrylate, 3-hydroxybutyl (meth)acrylate, and 4-hydroxybutyl (meth)acrylate.

The number of carbon atoms of the alkyl group contained in the hydroxyalkyl (meth)acrylate is preferably 1 to 10, more preferably 1 to 8, further preferably 1 to 6, and further more preferably 2 to 4, and the alkyl group may be a linear alkyl group or a branched alkyl group.

Examples of the epoxy-containing monomer include epoxy group-containing (meth)acrylates such as glycidyl (meth)acrylate, β-methylglycidyl (meth)acrylate, (3,4-epoxy cyclohexyl)methyl (meth)acrylate, and 3-epoxycyclo-2-hydroxypropyl (meth)acrylate; glycidyl crotonate, and allyl glycidyl ether.

Examples of the cycloaliphatic structure-containing monomer include cycloalkyl (meth)acrylates such as cyclopropyl (meth)acrylate, cyclobutyl (meth)acrylate, cyclopentyl (meth)acrylate, cyclohexyl (meth)acrylate, cycloheptyl (meth)acrylate, and cyclooctyl (meth)acrylate, isobornyl (meth)acrylate, and dicyclopentenyl (meth)acrylate.

As the resin (A2) used in one aspect of the present invention, a resin having a constitutional unit derived from adamantyl (meth)acrylate as the constitutional unit derived from the cycloaliphatic structure-containing monomer may be used. Such a resin corresponds to not only the resin (A2), but also the resin (A3) described below.

The resin (A2) used in one aspect of the present invention may have a constitutional unit derived from a monomer selected from the group consisting of an ester of a compound having two or more hydroxyl groups in the molecule, such as a dihydric or higher polyhydric alcohol, polyether diol, and polyester diol with a (meth)acrylic acid; an adduct of a compound having two or more epoxy groups in the molecule, exemplified by an epoxy resin, with a (meth)acrylic acid; and a condensate of a compound having two or more amino groups in the molecule with a (meth)acrylic acid.

Examples of such a monomer include (poly)alkylene glycol (derivative) di(meth)acrylates such as ethylene glycol di(meth)acrylate, diethylene glycol di(meth)acrylate, triethylene glycol di(meth)acrylate, propylene glycol di(meth)acrylate, dipropylene glycol di(meth)acrylate, tripropylene glycol di(meth)acrylate, butanediol di(meth)acrylate, trimethylolpropane di(meth)acrylate, trimethylolpropane tri(meth)acrylate, pentaerythritol tri(meth)acrylate, pentaerythritol tetra(meth)acrylate, tricyclodecane dimethanol di(meth)acrylate, trimethylolpropane tri(meth)acrylate, N,N′-methylenebis (meth)acrylamide, and di(meth)acrylate of an ethylene glycol adduct or a propylglycol adduct of bisphenol A; and epoxy (meth)acrylates such as a (meth)acrylic acid adduct of bisphenol A diglycidyl ether.

The weight average molecular weight (Mw) of the resin (A2) used in one aspect of the present invention is preferably 400 to 50,000, more preferably 1,000 to 40,000, further preferably 1,000 to 30,000, and further more preferably 1,000 to 25,000.

### [Resin (A3)]

The resin (A3) used in one aspect of the present invention is not particularly limited, and a known resin is used. A resin having (a3-1) a constitutional unit having an adamantane structure is used, and the constitutional unit is desirably a constitutional unit capable of being decomposed by an action of an acid to form an acid functional group. In view of the solubility in a solvent and the adhesiveness to a substrate, the resin (A3) used in one aspect of the present invention is practically preferably a copolymer having: (a3-2) a constitutional unit having a lactone structure, together with the constitutional unit (a3-1).

At least one of the hydrogen atoms to which carbon atoms constituting the adamantane structure in the constitutional unit (a3-1) are bonded may be replaced with a substituent R.

Also, at least one of the hydrogen atoms to which carbon atoms constituting the lactone structure in the constitutional unit (a3-2) are bonded may be replaced with the substituent R.

Examples of the substituent R include an alkyl group having 1 to 6 carbon atoms, a hydroxyalkyl group having 1 to 6 carbon atoms, a cycloalkyl group having 3 to 6 carbon atoms, a halogen atom (fluorine atom, chlorine atom, bromine atom, and iodine atom), a deuterium atom, a hydroxy group, an amino group, a nitro group, a cyano group, and a group represented by the following formula (i) or (ii).

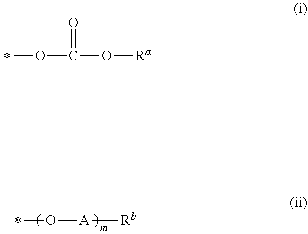

In the above formula (i) or (ii), R^{a} and R^{b} are each independently an alkyl group having 1 to 6 carbon atoms, a hydroxyalkyl group having 1 to 6 carbon atoms, or a cycloalkyl group having 3 to 6 carbon atoms.

m is an integer of 1 to 10, preferably an integer of 1 to 6, more preferably an integer of 1 to 3, and further preferably an integer of 1 to 2.

A is an alkylene group having 1 to 6 carbon atoms (preferably 1 to 4 carbon atoms, and more preferably 2 to 3).

Examples of the alkylene group include a methylene group, an ethylene group, an n-propylene group, an i-propylene group, a 1,4-butylene group, a 1,3-butylene group, a tetramethylene group, a 1,5-pentylene group, a 1,4-pentylene group, and a 1,3-pentylene group.

The resin (A3) used in one aspect of the present invention may have: (a3-1a) the constitutional unit having an adamantane structure substituted with a hydroxy group, which is a constitutional unit (a3-1), and in the resin (A3), the content of the constitutional unit (a3-1α) is preferably less than 50 mol %, more preferably less than 44 mol %, further preferably less than 39 mol %, and further more preferably less than 34 mol %, based on the total amount (100 mol %) of the constitutional unit of the resin (A3).

In one aspect of the present invention, the constitutional unit (a3-1) is preferably a constitutional unit (a3-1-1) represented by the following formula (a3-1-i) or a constitutional unit (a3-1-2) represented by the following formula (b2-1-ii).

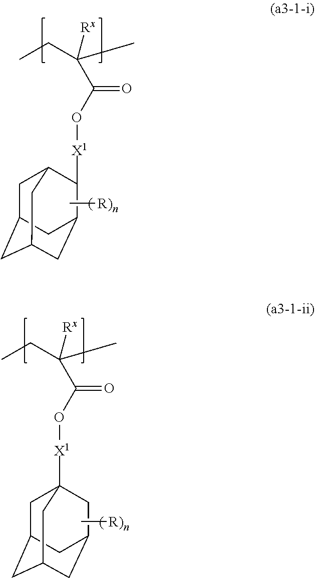

In the above formula, n is each independently an integer of 0 to 14, preferably an integer of 0 to 4, more preferably an integer of 0 to 2, and further preferably an integer of 0 to 1.

R^{x} is each independently a hydrogen atom or a methyl group.

R is each independently a substituent R that may be included in the adamantane structure, and is specifically as described above. R is preferably an alkyl group having 1 to 6 carbon atoms, and more preferably an alkyl group having 1 to 3 carbon atoms.

X¹ is each independently a single bond, an alkylene group having 1 to 6 carbon atoms, or a divalent linking group represented by any of the following formulas.

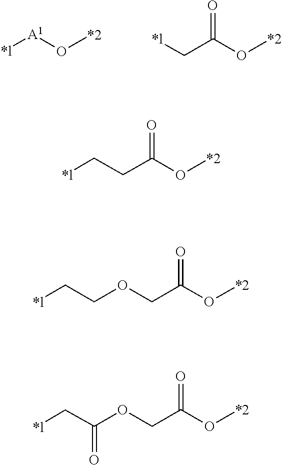

In the above formula, *1 represents a binding site with an oxygen atom in the above formula (a3-1-i) or (a3-1-ii), *2 represents a binding site with a carbon atom in the adamantane structure. A¹ represents an alkylene group having 1 to 6 carbon atoms.

In one aspect of the present invention, the constitutional unit (a3-2) is preferably any of (a3-2-1) a constitutional unit represented by the following formula (a3-2-i), (a3-2-2) a constitutional unit represented by the following formula (a3-2-ii), and (a3-2-3) a constitutional unit represented by the following formula (a3-2-iii).

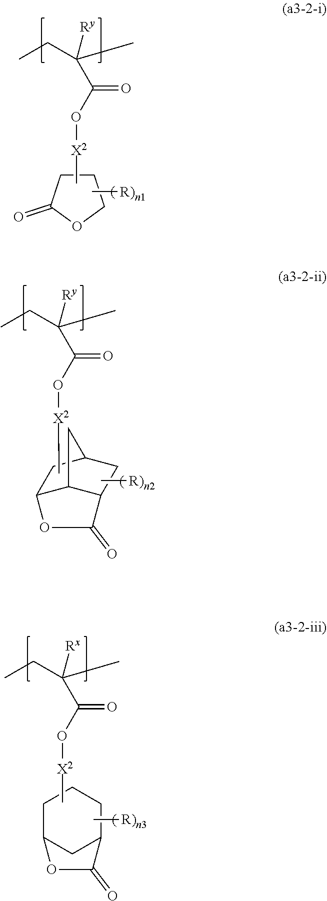

In the above formula, n1 is an integer of 0 to 5, preferably an integer of 0 to 2, and more preferably an integer of 0 to 1.

n2 is an integer of 0 to 9, preferably an integer of 0 to 2, and more preferably an integer of 0 to 1.

n3 is an integer of 0 to 9, preferably an integer of 0 to 2, and more preferably an integer of 0 to 1.

R^{y} is a hydrogen atom or a methyl group.

R is each independently a substituent R that may be included in the lactone structure, and is specifically as described above. R is preferably an alkyl group having 1 to 6 carbon atoms, and more preferably an alkyl group having 1 to 3 carbon atoms. When a plurality of R is present, the plurality of R may be the same groups or groups different from each other.

X² is a single bond, an alkylene group having 1 to 6 carbon atoms, or a divalent linking group represented by any of the following formulas.

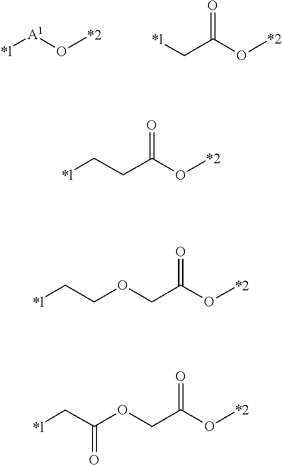

In the above formula, *1 represents a binding site with an oxygen atom in the above formula (a3-2-i), (a3-2-ii), or (a3-2-iii), and *2 represents a binding site with a carbon atom in the lactone structure. A¹ represents an alkylene group having 1 to 6 carbon atoms.

The resin (A³) used in one aspect of the present invention may have other constitutional units in addition to the constitutional units (a3-1) and (a3-2).

Examples of such other constitutional units include constitutional units derived from monomers such as alkyl (meth)acrylate; hydroxy group-containing monomer; epoxy group-containing monomer; a cycloaliphatic structure-containing monomer; an olefin such as ethylene, propylene, and isobutylene; halogenated olefins such as vinyl chloride and vinylidene chloride; diene monomers such as butadiene, isoprene, and chloroprene; styrene, α-methylstyrene, vinyl toluene, acrylonitrile, (meth)acrylamide, (meth) acrylonitrile, (meth)acryloylmorpholine, and N-vinyl pyrrolidone. The details of these monomers are the same as the description in the item of the resin (A²).

In the resin (A³) used in one aspect of the present invention, the total content of the constitutional units (a3-1) and (a3-2) is preferably 30 to 100 mol %, more preferably 50 to 100 mol %, further preferably 70 to 100 mol %, further more preferably 80 to 100 mol %, and particularly preferably 90 to 100 mol %, based on the total amount (100 mol %) of the constitutional unit of the resin (A²b).

The weight average molecular weight (Mw) of the resin (A³) used in one aspect of the present invention is preferably 400 to 50,000, more preferably 2,000 to 40,000, further preferably 3,000 to 30,000, and further more preferably 4,000 to 20,000.

The molecular weight distribution (Mw/Mn) of the resin (A³) is preferably 6.0 or less, more preferably 5.0 or less, further preferably 4.0 or less, and further more preferably 3.2 or less, and preferably 1.01 or more, more preferably 1.05 or more, and further preferably 1.1 or more.

### [Resin (A⁴)]

The resin (A⁴) used in one aspect of the present invention in not particularly limited, as long as it is a resin having any two or more constitutional units of (a2-1) the constitutional unit derived from a phenolic hydroxyl group-containing compound, (a2-2) the constitutional unit capable of being decomposed by an action of an acid, a base, or heat to form an acid functional group, (a3-1) the constitutional unit having an adamantane structure, and (a3-2) the constitutional unit having a lactone structure (provided that, the resin (A²) and the resin (A³) are excluded), and a known resin can be used. Example thereof that can be used include resins exemplified in a book “40 years of lithography technology”, International Publication No. WO 2014-175275, International Publication No. WO 2015-115613, International Publication No. WO 2020-137935, International Publication No. WO 2021-029395, and International Publication No. WO 2021-029396.

The weight average molecular weight (Mw) of the resin (A⁴) used in one aspect of the present invention is preferably 400 to 50,000, more preferably 2,000 to 40,000, further preferably 3,000 to 30,000, and further more preferably 4,000 to 20,000.

The molecular weight distribution (Mw/Mn) of the resin (A⁴) is preferably 6.0 or less, more preferably 5.0 or less, further preferably 4.0 or less, and further more preferably 3.2 or less, and preferably 1.01 or more, more preferably 1.05 or more, and further preferably 1.1 or more.

### <Component (B): Solvent>

The resist composition of one aspect of the present invention contains: (B) a solvent containing (B1) a compound represented by the following general formula (b-1).

The compound (B1) may be used singly or in combination of two or more

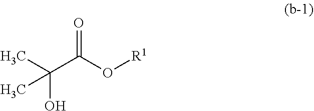

In the above formula (b-1), R¹ is an alkyl group having 1 to 10 carbon atoms. The alkyl group may be a linear alkyl group or a branched alkyl group.

Examples of the alkyl group capable of being selected as R¹ include a methyl group, an ethyl group, an n-propyl group, an i-propyl group, an n-butyl group, an i-butyl group, an s-butyl group, or a t-butyl group, a pentyl group, a hexyl group, a heptyl group, an octyl group, a 2-ethylhexyl group, a nonyl group, and a decyl group.

Among these, R¹ in the general formula (b-1) is preferably a methyl group, an ethyl group, an n-propyl group, an i-propyl group, an n-butyl group, an i-butyl group, an s-butyl group, or a t-butyl group, in one aspect of the present invention, more preferably an ethyl group, an n-propyl group, an i-propyl group, an n-butyl group, an i-butyl group, an s-butyl group, or a t-butyl group, further preferably an n-propyl group, an i-propyl group, an n-butyl group, an i-butyl group, an s-butyl group, or a t-butyl group, and further more preferably an i-propyl group, an n-butyl group, or an i-butyl group.

In the resist composition of one aspect of the present invention, (B2) a solvent other than compound (B1) is preferably contained as the component (B).

Examples of the solvent (B2) include lactones such as γ-butyrolactone; ketones such as acetone, methyl ethyl ketone, cyclohexanone, methyl-n-pentyl ketone, methyl isopentyl ketone, and 2-heptanone; polyhydric alcohols such as ethylene glycol, diethylene glycol, propylene glycol, and dipropylene glycol; compounds having an ester bond such as ethylene glycol monoacetate, diethylene glycol monoacetate, propylene glycol monoacetate, and dipropylene glycol monoacetate; compounds having an ether bond, such as mono alkyl ethers such as monomethyl ether, monoethyl ether, monopropyl ether, and monobutyl ether or monophenyl ethers of the polyhydric alcohol or the compounds having an ester bond such as 1-methoxy 2-propanol; cyclic ethers such as dioxane, and esters other than the compound (B1), such as methyl lactate, ethyl lactate, methyl acetate, ethyl acetate, butyl acetate, methyl pyruvate, ethyl pyruvate, methyl α-methoxyisobutyrate, methyl β-methoxyisobutyrate, ethyl 2-ethoxyisobutyrate, methyl methoxypropionate, ethyl ethoxypropionate, methyl-formyloxyisobutyrate, methyl β-formyl oxyisobutyrate, and methyl 3-hydroxyisobutyrate; aromatic organic solvents such as anisole, ethylbenzyl ether, cresyl methyl ether, diphenyl ether, dibenzyl ether, phenetole, butyl phenyl ether, ethylbenzene, diethylbenzene, pentylbenzene, isopropylbenzene, toluene, xylene, cymene, and mesitylene; and dimethylsulfoxide (DMSO).

These solvents (B2) may be used singly or in combination of two or more thereof.

However, in the resist composition of the present invention, the content of the compound (B1) in the component (B) is preferably 20 to 100% by mass, more preferably 30 to 100% by mass, further preferably 50 to 100% by mass, further more preferably 60 to 100% by mass, and particularly preferably 70 to 100% by mass, based on the total amount (100% by mass) of the component (B) contained in the resist composition, in view of producing a photoresist material capable of forming a thick resist film.

The component (B) used in one aspect of the present invention preferably contains one or more selected from the group consisting of methyl α-methoxyisobutyrate, methyl α-formyloxyisobutyrate, methyl α-acetyloxyisobutyrate, methyl 3-hydroxyisobutyrate, and 1-methoxy-2-propanol as the solvent (B2), in view of the solubility of the acid generating agent used in the resist composition. It is preferable to contain methyl α-methoxyisobutyrate, in view of the solubility of the resin used in the resist composition. It is preferable to contain methyl α-formyloxyisobutyrate or methyl α-acetyloxyisobutyrate, in view of forming a thick soluble resist film from the resin used in the resist composition. It is preferable to contain methyl 3-hydroxyisobutyrate, in view of obtaining a rectangular resist pattern. It is preferable to contain 1-methoxy-2-propanol, in view of obtaining a resist film having high in-plane uniformity. The method for mixing methyl α-methoxyisobutyrate, methyl α-formyloxyisobutyrate, methyl «-acetyloxyisobutyrate, methyl 3-hydroxyisobutyrate, or 1-methoxy-2-propanol is not particularly limited, and they can be contained by either a method including adding methyl α-methoxyisobutyrate, methyl «-formyloxyisobutyrate, methyl 3-hydroxyisobutyrate, or 1-methoxy-2-propanol to the compound (B1), or a method including mixing the component (B) by forming any of them as a by-product or incorporating any of them in the manufacturing process of the compound (B1).

The content of the solvent (B2) is not limited, and is preferably less than 100% by mass based on the total amount (100% by mass) of the compound (B1), in view of shortening the drying time of the coating film to improve productivity. The content is preferably 70% by mass or less, and more preferably 60% by mass or less, 50% by mass or less, 40% by mass or less, 30% by mass or less, 20% by mass or less, 10% by mass or less, 5% by mass or less, 1% by mass or less, in view of increasing the solvency of the solvent while ensuring a moderate drying time, and is further preferably 0.1% by mass or less, and particularly preferably 0.01% by mass or less. The content of the solvent (B2) is preferably 0.0001% by mass or more in view of improving the storage stability of the resist composition, more preferably 0.001% by mass or more in view of improving the solubility of the active component of the resist composition, and further preferably 0.01% by mass or more in view of suppressing the defect of the resist film.

The content of methyl «-methoxyisobutyrate, methyl α-formyloxyisobutyrate, methyl α-acetyloxyisobutyrate, methyl 3-hydroxyisobutyrate, or 1-methoxy-2-propanol is not limited, and is preferably less than 100% by mass, more preferably 70% by mass or less, 60% by mass or less, 50% by mass or less, 40% by mass or less, 30% by mass or less, 20% by mass or less, 10% by mass or less, 5% by mass or less, and 1% by mass or less, further preferably 0.1% by mass or less, and particularly preferably 0.01% by mass or less, based on the total amount (100% by mass) of the resist composition, in view of shortening the drying time of the coating film to improve productivity. The content thereof is preferably 0.0001% by mass or more in view of improving the storage stability of the resist composition, more preferably 0.001% by mass or more in view of improving the solubility of the active component of the resist composition, and further preferably 0.01% by mass or more in view of suppressing the defect of the resist film.

The content of methyl α-methoxyisobutyrate, methyl α-formyloxyisobutyrate, methyl α-acetyloxyisobutyrate, methyl 3-hydroxyisobutyrate, or 1-methoxy-2-propanol is preferably 100% by mass or less, more preferably 70% by mass or less, 60% by mass or less, 50% by mass or less, 40% by mass or less, 30% by mass or less, 20% by mass or less, 10% by mass or less, 5% by mass or less, and 1% by mass or less, further preferably 0.1% by mass or less, and particularly preferably 0.01% by mass or less, based on the total amount (100% by mass) of the compound (B1), in view of improving productivity by shortening the drying time of the resist composition. The content thereof is preferably 0.0001% by mass or more in view of improving the storage stability of the resist composition, more preferably 0.001% by mass or more in view of improving the solubility of the active component of the resist auxiliary film composition, and further preferably 0.01% by mass or more in view of suppressing the defect of the resist film.

The content of 1-methoxy-2-propanol is preferably 1 to 98% by mass, and more preferably 16 to 98% by mass, based on the total amount (100% by mass) of the resist composition, in view of the in-plane uniformity of the coating film. In addition, the content of 1-methoxy-2-propanol is also preferably 1 to 99% by mass, and also more preferably 30 to 99% by mass, based on the total amount (100% by mass) of the compound (B1).

The component (B) used in one aspect of the present invention preferably contains one or more selected from the group consisting of methyl α-formyloxyisobutyrate, methyl α-acetyloxyisobutyrate, and methyl 3-hydroxyisobutyrate, as the solvent (B2).

In the resist composition of the present invention, the content of the component (B) may be appropriately set depending on the application, and may be 50% by mass or more, 54% by mass or more, 58% by mass or more, 60% by mass or more, 65% by mass or more, 69% by mass or more, 74% by mass or more, 77% by mass or more, 80% by mass or more, 82% by mass or more, 84% by mass or more, 88% by mass or more, 90% by mass or more, 94% by mass or more, or 97% by mass or more, based on the total amount (100% by mass) of the resist composition.

The upper limit value of the content of the component (B) may be appropriately set according to the content of the component (A), and the content may be 99% by mass or less, 98% by mass or less, 96% by mass or less, 93% by mass or less, 91% by mass or less, 86% by mass or less, 81% by mass or less, 76% by mass or less, 71% by mass or less, 66% by mass or less, or 61% by mass or less, based on the total amount (100% by mass) of the resist composition.

The range of the content of the component (B) can be specified by any combination of an upper limit value and a lower limit value appropriately selected from the options each mentioned above.

<Component (C): Additive Selected from Photosensitizer and Acid Generating Agent>

The resist composition of one aspect of the present invention preferably contains: (C) at least one additive selected from the group consisting of a photosensitizer and an acid generating agent.

The component (C) may be used singly or in combination of two or more thereof.

In the resist composition of one aspect of the present invention, the content of the component (C) is preferably 0.01 to 80 parts by mass, more preferably 0.05 to 65 parts by mass, further preferably 0.1 to 50 parts by mass, and further more preferably 0.5 to 30 parts by mass per 100 parts by mass of the resin (A) contained in the resist composition.

Hereinafter, the photosensitizer and the acid generating agent contained as the component (C) will be described.

### [Photosensitizer]

The photosensitizer that may be selected as the component (C) is not particularly limited, as long as it is typically used as the photosensitive component in a positive type resist composition.

The photosensitizers may be used singly or in combination of two or more thereof.

Examples of the photosensitizer used in one aspect of the present invention include a reactant of acid chloride and a compound having a functional group condensable with the acid chloride (such as a hydroxyl group and an amino group).

Examples of the acid chloride include naphthoquinonediazidosulfonic acid chloride and benzoquinonediazidosulfonic acid chloride, and specific examples thereof include 1,2-naphthoquinonediazido-5-sulfonyl chloride and 1,2-naphthoquinonediazido-4-sulfonyl chloride.

Examples of the compound having a functional group condensable with the acid chloride include hydroxybenzophenones such as hydroquinone, resorcin, 2,4-dihydroxybenzophenone, 2,3,4-trihydroxybenzophenone, 2,4,6-trihydroxybenzophenone, 2,4,4′-trihydroxybenzophenone, 2,3,4,4′-tetrahydroxybenzophenone, 2,2′,4,4′-tetrahydroxybenzophenone, and 2,2′,3,4,6′-pentahydroxybenzophenone; hydroxyphenylalkanes such as bis(2,4-dihydroxyphenyl)methane, bis(2,3,4-trihydroxyphenyl)methane, and bis(2,4-dihydroxyphenyl)propane; and hydroxytriphenylmethanes such as 4,4′,3″,4″-tetrahydroxy-3,5,3′,5′-tetramethyltriphenylmethane, and 4,4′,2″,3″,4″-pentahydroxy-3,5,3′,5′-tetramethyltriphenylmethane.

As the photosensitizer used in one aspect of the present invention, commercial products such as “DTEP-350” (a diazonaphthoquinone photosensitizer manufactured by DAITO CHEMIX Co., Ltd.) may be used.

### [Acid Generating Agent]

The acid generating agent that may be selected as the component (c) may be a compound capable of directly or indirectly generating an acid by irradiation with radiation such as a visible light, an ultraviolet, an excimer laser, an electron beam, an extreme ultraviolet (EUV), an X-ray, and an ion beam.

Specifically, as the suitable acid generating agent, a compound represented by any of the following general formulas (c-1) to (c-8) is preferable.

### (Compound Represented by General Formula (c-1))

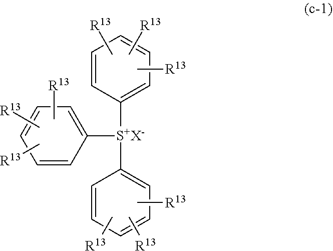

In the above formula (c-1), R¹³ is each independently a hydrogen atom, a linear, branched, or cyclic alkyl group, a linear, branched, or cyclic alkoxy group, a hydroxyl group, or a halogen atom.

X⁻ is a sulfonic acid ion or halide ion having an alkyl group, an aryl group, a halogen-substituted alkyl group, or a halogen-substituted aryl group.

The compound represented by the general formula (c-1) is preferably at least one selected from the group consisting of triphenylsulfonium trifluoromethanesulfonate, triphenylsulfonium nonafluoro-n-butanesulfonate, diphenyltolylsulfonium nonafluoro-n-butanesulfonate, triphenylsulfonium perfluoro-n-octanesulfonate, diphenyl-4-methylphenylsulfonium trifluoromethanesulfonate, di-2,4,6-trimethylphenylsulfonium trifluoromethanesulfonate, diphenyl-4-t-butoxyphenylsulfonium trifluoromethanesulfonate, diphenyl-4-t-butoxyphenylsulfonium nonafluoro-n-butanesulfonate, diphenyl-4-hydroxyphenylsulfonium trifluoromethanesulfonate, bis(4-fluorophenyl)-4-hydroxyphenylsulfonium trifluoromethanesulfonate, diphenyl-4-hydroxyphenylsulfonium phenylsulfonium trifluoromethanesulfonate, tri(4-methoxyphenyl) sulfonium nonafluoro-n-butanesulfonate, bis(4-hydroxyphenyl)-trifluoromethanesulfonate, tri(4-fluorophenyl) sulfonium trifluoromethanesulfonate, triphenylsulfonium p-toluene sulfonate, triphenylsulfonium benzenesulfonate, diphenyl-2,4,6-trimethylphenyl-p-toluene sulfonate, diphenyl-2,4,6-trimethylphenylsulfonium-2-trifluoromethylbenzenesulfonate, diphenyl-2,4,6-trimethylphenylsulfonium-4-trifluoromethylbenzenesulfonate, diphenyl-2,4,6-trimethylphenylsulfonium-2,4-difluorobenzenesulfonate, diphenyl-2,4,6-trimethylphenylsulfonium hexafluorobenzenesulfonate, diphenylnaphthylsulfonium trifluoromethanesulfonate, diphenyl-4-hydroxyphenylsulfonium-p-toluene sulfonate, triphenylsulfonium 10-camphorsulfonate, diphenyl-4-hydroxyphenylsulfonium 10-camphorsulfonate, and cyclo(1,3-perfluoropropanedisulfone) imidate.

### Compound Represented by General Formula (c-2)

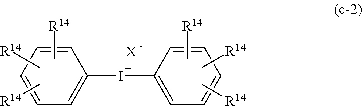

In the above formula (c-2), R¹⁴ is each independently a hydrogen atom, a linear, branched, or cyclic alkyl group, a linear, branched, or cyclic alkoxy group, a hydroxyl group, or a halogen atom.

X⁻ is a sulfonic acid ion or halide ion having an alkyl group, an aryl group, a halogen-substituted alkyl group, or a halogen-substituted aryl group.

The compound represented by the general formula (c-2) is preferably at least one selected from the group consisting of bis(4-t-butylphenyl)iodonium trifluoromethanesulfonate, bis(4-t-butylphenyl)iodonium nonafluoro-n-butanesulfonate, bis(4-t-butylphenyl)iodonium perfluoro-n-octanesulfonate, bis(4-t-butylphenyl)iodonium p-toluene sulfonate, bis(4-t-butylphenyl)iodonium benzenesulfonate, bis(4-t-butylphenyl)iodonium-2-trifluoromethylbenzenesulfonate, bis(4-t-butylphenyl)iodonium-4-trifluoromethylbenzenesulfonate, bis(4-t-butylphenyl)iodonium-2,4-difluorobenzenesulfonate, bis(4-t-butylphenyl)iodonium hexafluorobenzenesulfonate, bis(4-t-butylphenyl)iodonium 10-camphorsulfonate, diphenyliodonium trifluoromethanesulfonate, diphenyliodonium nonafluoro-n-butanesulfonate, diphenyliodonium perfluoro-n-octanesulfonate, diphenyliodonium p-toluene sulfonate, diphenyliodonium benzenesulfonate, diphenyliodonium 10-camphorsulfonate, diphenyliodonium-2-trifluoromethylbenzenesulfonate, diphenyliodonium-4-trifluoromethylbenzenesulfonate, diphenyliodonium-2,4-difluorobenzenesulfonate, diphenyliodonium hexafluorobenzenesulfonate, di(4-trifluoromethylphenyl)iodonium trifluoromethanesulfonate, di(4-trifluoromethylphenyl)iodonium nonafluoro-n-butanesulfonate, di(4-trifluoromethylphenyl)iodonium perfluoro-n-octanesulfonate, di(4-trifluoromethylphenyl)iodonium p-toluene sulfonate, di(4-trifluoromethylphenyl)iodonium benzenesulfonate, and di(4-trifluoromethylphenyl)iodonium 10-camphorsulfonate.

### Compound Represented by General Formula (c-3)

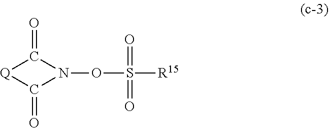

In the above formula (c-3), Q is an alkylene group, an arylene group, or an alkoxylene group. R¹⁵ is an alkyl group, an aryl group, a halogen-substituted alkyl group, or a halogen-substituted aryl group.

The compound represented by the general formula (c-3) is preferably at least one selected from the group consisting of N-(trifluoromethylsulfonyloxy) succinimide, N-(trifluoromethylsulfonyloxy) phthalimide, N-(trifluoromethylsulfonyloxy)diphenylmaleimide, N-(trifluoromethylsulfonyloxy)bicyclo[2.2.1]hept-5-ene-2,3-dicarboxyimido, N-(trifluoromethylsulfonyloxy) naphthylimido, N-(10-camphorsulfonyloxy) succinimide, N-(10-camphorsulfonyloxy) phthalimide, N-(10-camphorsulfonyloxy)diphenylmaleimide, N-(10-camphorsulfonyloxy)bicyclo[2.2.1]hept-5-ene-2,3-dicarboxyimido, N-(10-camphorsulfonyloxy) naphthylimido, N-(n-octanesulfonyloxy)bicyclo[2.2.1]hept-5-ene-2,3-dicarboxyimido, N-(n-octanesulfonyloxy) naphthylimido, N-(p-toluenesulfonyloxy)bicyclo[2.2.1]hept-5-ene-2,3-dicarboxyimido, N-(p-toluenesulfonyloxy) naphthylimido, N-(2-trifluoromethylbenzenesulfonyloxy)bicyclo[2.2.1]hept-5-ene-2,3-dicarboxyimido, N-(2-trifluoromethylbenzenesulfonyloxy) naphthylimido, N-(4-trifluoromethylbenzenesulfonyloxy)bicyclo[2.2.1]hept-5-ene-2,3-dicarboxyimido, N-(4-trifluoromethylbenzenesulfonyloxy) naphthylimido, N-(perfluorobenzenesulfonyloxy)bicyclo[2.2.1]hept-5-ene-2,3-dicarboxyimido, N-(perfluorobenzenesulfonyloxy) naphthylimido, N-(1-naphthalene sulfonyloxy)bicyclo[2.2.1]hept-5-ene-2,3-dicarboxyimido, N-(1-naphthalene sulfonyloxy) naphthylimido, N-(nonafluoro-n-butanesulfonyloxy)bicyclo[2.2.1]hept-5-ene-2,3-dicarboxyimido, N-(nonafluoro-n-butanesulfonyloxy) naphthylimido, N-(perfluoro-n-octanesulfonyloxy)bicyclo[2.2.1]hept-5-ene-2,3-dicarboxyimido, and N-(perfluoro-n-octanesulfonyloxy) naphthylimido.

### Compound Represented by General Formula (c-4)

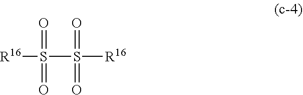

In the above formula (c-4), R¹⁶ is each independently a linear, branched, or cyclic alkyl group, an aryl group, a heteroaryl group, or an aralkyl group, and at least one hydrogen of these groups may be replaced with an arbitrary substituent.

The compound represented by the general formula (c-4) is preferably at least one selected from the group consisting of diphenyl disulfone, di(4-methylphenyl)disulfone, dinaphthyldisulfone, di(4-t-butylphenyl)disulfone, di(4-hydroxyphenyl)disulfone, di(3-hydroxynaphthyl)disulfone, di(4-fluorophenyl)disulfone, di(2-fluorophenyl)disulfone, and di(4-trifluoromethylphenyl)disulfone.

### Compound Represented by General Formula (c-5)

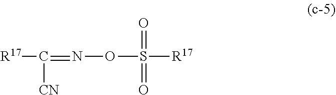

In the above formula (c-5), R¹⁷ is each independently a linear, branched, or cyclic alkyl group, an aryl group, a heteroaryl group, or an aralkyl group, and at least one hydrogen of these groups may be replaced with an arbitrary substituent.

The compound represented by the general formula (c-5) is preferably at least one selected from the group consisting of α-(methylsulfonyloxyimino)-phenylacetonitrile, α-(methylsulfonyloxyimino)-4-methoxyphenylacetonitrile, α-(trifluoromethylsulfonyloxyimino)-phenylacetonitrile, (trifluoromethylsulfonyloxyimino)-4-methoxyphenylacetonitrile, α-(ethylsulfonyloxyimino)-4-methoxyphenylacetonitrile, α-(propylsulfonyloxyimino)-4-methylphenylacetonitrile, and α-(methylsulfonyloxyimino)-4-bromophenylacetonitrile.

### Compound Represented by General Formula (c-6)

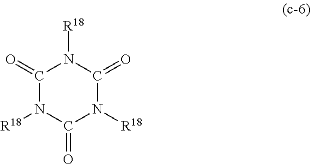

In the above formula (c-6), R¹⁸ is each independently a halogenated alkyl group having one or more chlorine atoms and one or more bromine atoms. The number of carbon atoms of the halogenated alkyl group is preferably 1 to 5.

### Compound Represented by General Formulas (c-7) and (c-8)

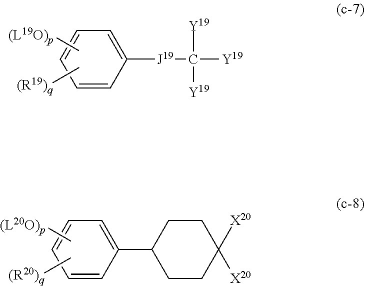

In the above formulas (c-7) and (c-8), R¹⁹ and R²⁰ are each independently an alkyl group having 1 to 3 carbon atoms (such as a methyl group, an ethyl group, an n-propyl group, or an i-propyl group), a cycloalkyl group having 3 to 6 carbon atoms (such as a cyclopentyl group or a cyclohexyl group), an alkoxyl group having 1 to 3 carbon atoms (such as a methoxy group, an ethoxy group, or a propoxy group), or an aryl group having 6 to 10 carbon atoms (a phenyl group, a toluyl group, or a naphthyl group), and an aryl group having 6 to 10 carbon atoms is preferable.

L¹⁹ and L²⁰ are each independently an organic group having a 1,2-naphthoquinonediazido group, and are specifically preferably a 1,2-quinonediazidosulfonyl group such as a 1,2-naphthoquinonediazido-4-sulfonyl group, a 1,2-naphthoquinonediazido-5-sulfonyl group, and a 1,2-naphthoquinonediazido-6-sulfonyl group, and more preferably a 1,2-naphthoquinonediazido-4-sulfonyl group or a 1,2-naphthoquinonediazido-5-sulfonyl group.

p is an integer of 1 to 3, q is an integer of 0 to 4, and 1≤ p+q≤5.

J¹⁹ is a single bond, an alkylene group having 1 to 4 carbon atoms, a cycloalkylene group having 3 to 6 carbon atoms, a phenylene group, a group represented by the following formula (c-7-i), a carbonyl group, an ester group, an amide group, or —O—.

Y¹⁹ is a hydrogen atom, an alkyl group having 1 to 6 carbon atoms, or an aryl group having 6 to 10 carbon atoms, and X²⁰ is each independently a group represented by the following formula (c-8-i).

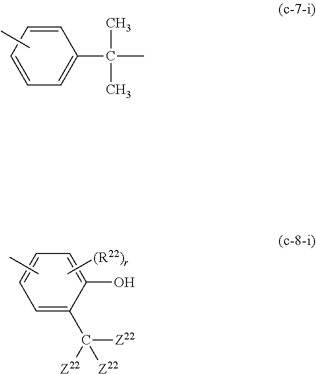

In the above formula (c-8-i), Z²² is each independently an alkyl group having 1 to 6 carbon atoms, a cycloalkyl group having 3 to 6 carbon atoms, or an aryl group having 6 to 10 carbon atoms. R²² is each independently an alkyl group having 1 to 6 carbon atoms, a cycloalkyl group having 3 to 6 carbon atoms, or an alkoxyl group having 1 to 6 carbon atoms, and r is an integer of 0 to 3.

As the acid generating agent used in one aspect of the present invention, other acid generating agents may be used in addition to the compound represented by any of the general formulas (c-1) to (c-8).

Examples of such other acid generating agents include bissulfonyldiazomethanes such as bis(p-toluenesulfonyl)diazomethane, bis(2,4-dimethylphenylsulfonyl)diazomethane, bis(tert-butylsulfonyl)diazomethane, bis(n-butylsulfonyl)diazomethane, bis(isobutylsulfonyl)diazomethane, bis(n-propylsulfonyl)diazomethane, bis(isopropylsulfonyl)diazomethane, bis(cyclohexylsulfonyl)diazomethane, bis(isopropylsulfonyl)diazomethane, 1,3-bis(cyclohexylsulfonylazomethylsulfonyl)propane, 1,4 bis(phenylsulfonylazomethylsulfonyl)butane, 1,6-bis(phenylsulfonylazomethylsulfonyl)hexane, and 1,10-bis(cyclohexylsulfonylazomethylsulfonyl)decane; and halogen-containing triazine derivatives such as 2-(4-methoxyphenyl)-4,6-(bistrichloromethyl)-1,3,5-triazine, 2-(4-methoxynaphthyl)-4,6-(bistrichloromethyl)-1,3,5-triazine, tris(2,3-dibromopropyl)-1,3,5-triazine, and tris(2,3-dibromopropyl) isocyanurate.

### <Other Additives>

The resist composition of one aspect of the present invention may contain other components in addition to the aforementioned components (A) to (C).

Examples of other components include one or more selected from the group consisting of an acid cross-linking agent, an acid diffusion controlling agent, a dissolution accelerator, a dissolution controlling agent, a sensitizing agent, a surfactant, and an organic carboxylic acid or phosphorus oxoacid or a derivative thereof.

Each content of these other components is appropriately selected depending on the type of the component or the resin (A), and is preferably 0.001 to 100 parts by mass, more preferably 0.01 to 70 parts by mass, further preferably 0.1 to 50 parts by mass, and further more preferably 0.3 to 30 parts by mass, per 100 parts by mass of the resin (A) contained in the resist composition.

### (Acid Cross-Linking Agent)

The acid cross-linking agent may be a compound having a cross-linking group capable of cross-linking with the resin (A), and is appropriately selected depending on the type of the resin (A).

Examples of the acid cross-linking agent used in one aspect of the present invention include methylol group-containing compounds such as a methylol group-containing melamine compound, a methylol group-containing benzoguanamine compound, a methylol group-containing urea compound, a methylol group-containing glycoluril compound, and a methylol group-containing phenolic compound; alkoxyalkyl group-containing compounds such as an alkoxyalkyl group-containing melamine compound, an alkoxyalkyl group-containing benzoguanamine compound, an alkoxyalkyl group-containing urea compound, an alkoxyalkyl group-containing glycoluril compound, and an alkoxyalkyl group-containing phenolic compound; carboxymethyl group-containing compounds such as a carboxymethyl group-containing melamine compound, a carboxymethyl group-containing benzoguanamine compound, a carboxymethyl group-containing urea compound, a carboxymethyl group-containing glycoluril compound, and a carboxymethyl group-containing phenolic compound; and epoxy compounds such as a bisphenol An epoxy compound, a bisphenol F epoxy compound, a bisphenol S epoxy compound, a novolac resin epoxy compound, a resol resin epoxy compound, and a poly(hydroxystyrene) epoxy compound.

These acid cross-linking agents may be used singly or in combination of two or more thereof.

### (Acid Diffusion Controlling Agent)

The acid diffusion controlling agent is an additive that functions to, for example, control diffusion in the resist film of an acid generated from the acid generating agent by irradiation with radiation to inhibit an unpleasant chemical reaction in an unexposed region.

Examples of the acid diffusion controlling agent used in one aspect of the present invention include, but are not particularly limited to, radiation decomposable basic compounds such as a nitrogen atom-containing basic compound, a basic sulfonium compound, and a basic iodonium compound.

These acid diffusion controlling agents may be used singly or in combination of two or more thereof.

### (Dissolution Accelerator)

The dissolution accelerator is an additive that functions to increase the solubility of the resin (A) in a developer to moderately increase the dissolution rate of the resin (A) in development.

Examples of the dissolution accelerator used in one aspect of the present invention include, but are not particularly limited to, phenolic compounds such as bisphenols and tris(hydroxyphenyl)methane.

These dissolution accelerators may be used singly or in combination of two or more thereof.

### (Dissolution Controlling Agent)

The dissolution controlling agent is an additive that functions to control the solubility of the resin (A) to moderately reduce the dissolution rate in development if the solubility thereof in the developer is too high.

Examples of the dissolution controlling agent used in one aspect of the present invention include, but are not particularly limited to, aromatic hydrocarbons such as phenanthrene, anthracene, and acenaphthene; ketones such as acetophenone, benzophenone, and phenylnaphthylketone; and sulfones such as methyl phenyl sulfone, diphenyl sulfone, and dinaphthyl sulfone.

These dissolution controlling agents may be used singly or in combination of two or more thereof.

### (Sensitizing Agent)

The sensitizing agent is an additive that functions to absorb the energy of radiation irradiated and transmit the energy to the acid generating agent to thereby increasing the amount of acid generated, and may improve the apparent sensibility of the resist.

Examples of the sensitizing agent used in one aspect of the present invention include benzophenones, biacetyls, pyrenes, phenothiazines, and fluorenes. These sensitizing agents may be used singly or in combination of two or more thereof.

### (Surfactant)

The surfactant is an additive that functions to improve, for example, the applicability, striation, and developability of the resist.

The surfactant used in one aspect of the present invention may be any of an anionic surfactant, a cationic surfactant, a nonionic surfactant, and an amphoteric surfactant, and a nonionic surfactant is preferable. Examples of the nonionic surfactant include polyoxyethylene higher alkyl ethers, polyoxyethylene higher alkylphenyl ethers, and higher fatty acid diesters of polyethylene glycol. These surfactants may be used singly or in combination of two or more thereof.

### (Organic Carboxylic Acid or Phosphorus Oxoacid or Derivative Thereof)

The organic carboxylic acid or phosphorus oxoacid or a derivative thereof is an additive that functions to, for example, prevent deterioration in sensibility and improve the resist pattern shape and stability in post exposure delay.

Examples of the organic carboxylic acid used in one aspect of the present invention include, but are not particularly limited to, malonic acid, citric acid, malic acid, succinic acid, benzoic acid, and salicylic acid. Examples of the phosphorus oxoacid or a derivative thereof include phosphoric acids or derivatives such as esters thereof, such as phosphoric acid, phosphoric acid di-n-butyl ester, and phosphoric acid diphenyl ester; phosphonic acid or derivatives such as esters thereof, such as phosphonic acid, phosphonic acid dimethyl ester, phosphonic acid di-n-butyl ester, phenylphosphonic acid, phosphonic acid diphenyl ester, and phosphonic acid dibenzyl ester; and phosphinic acid and derivatives such as esters thereof, such as phosphinic acid and phenylphosphinic acid.

These may be used singly or in combination of two or more.

### (Other Components)

The resist composition of one aspect of the present invention may contain a dye, a pigment, an adhesion aid, a halation preventing agent, a storage stabilizing agent, a defoaming agent, a shape improver, or the others, in addition to the aforementioned other components.

### [Method for Forming Resist Film]

As mentioned above, the resist composition of one aspect of the present invention can form a thick resist film suitable for the manufacture of various devices, although the content of the active component including the resin is limited to a predetermined value or less.

The method for forming a resist film is not particularly limited, and, for example, the method may include the following step (1), and preferably further including steps (2) to (3).
Step (1): a step of applying the aforementioned resist composition of one aspect of the present invention on a substrate to form a coating film.
Step (2): a step of performing heat treatment after step (1).
Step (3): a step of forming a resist pattern.

### <Step (1)>

In step (1), examples of the substrate on which a coating film is formed include, but are not particularly limited to, a substrate for an electronic component and a substrate for an electronic component on which a predetermined wiring pattern is formed. More specific examples thereof include metal substrates such as silicon wafers, copper, chrome, iron, and aluminum, and glass substrates. Examples of the material of wiring pattern include, but are not particularly limited to, copper, aluminum, nickel, and gold.

The substrate used in one aspect of the present invention may have, if necessary, an underlayer film formed from a material selected from an organic material and an inorganic material on the surface where the coating film is formed. When such a substrate with an underlayer film is used, the coating film is formed on the underlayer film.

Examples of the underlayer film forming material for forming the underlayer film include the composition for forming an underlayer film described in International Publication No. WO 2016/021511.

The substrate used in one aspect of the present invention may be subjected to, if necessary, surface treatment by applying a pre-wetting agent to the surface where the coating film is formed.

Typically, a substantial amount of a resist composition is scattered from the outer periphery, where the circumferential speed is significantly larger than that at the center position, and an increase in the consumption of the resist composition is problematic. For this problem, the application of the pre-wetting agent to the surface of the substrate enables the resist composition to be easily diffused on the substrate, so that the amount of the resist composition to be supplied can be reduced.

Examples of the pre-wetting agent include cyclohexanone, ethyl lactate, and methyl 3-methoxypropionate.

Specific examples of the surface treatment method involving use of a pre-wetting agent include, but are not particularly limited to, the method described in Japanese unexamined Patent Application Publication No. 2004-39828.

As the application method for applying the resist composition to the substrate, a known method can be appropriately used, and examples thereof include spin coating, flow coating, and roll coating. As described above, the resist composition of one aspect of the present invention can form a thick coating film by these application methods.

### <Step (2)>

In one aspect of the present invention, the step of performing heat treatment is preferably carried out as step (2), after step (1). By performing heat treatment, the adhesiveness between the substrate and the resist film can be improved.

The heating temperature of the heat treatment in this step is appropriately set according to the composition of the resist composition, and is preferably 20 to 250° C., and more preferably 20 to 150° C.

### <Step (3)>

Step (3) is a step of forming a predetermined resist pattern by exposing the formed resist film via a desired mask pattern.

Examples of radiation irradiated during exposure include ultraviolet rays typified by visible light, g-line (wavelength: 436 nm), and i-line (wavelength: 365 nm); far ultraviolet rays typified by ArF excimer laser (wavelength: 193 nm) and KrF excimer laser (wavelength: 248 nm); X-rays typified by excimer laser, electron beam, extreme ultraviolet (EUV), and synchrotron radiation; and ion beams.

In view of stably forming a more precise fine pattern in the exposure, heat treatment is preferably performed after irradiation with radiation. The heating temperature of the heat treatment is preferably 20 to 250° C., and more preferably 20 to 150° C.

Then, by developing the exposed resist film with a developer, a predetermined resist pattern can be formed.

As the developer to be used, a solvent having a solubility parameter (SP value) close to that of the resin (A) contained in the resist composition is preferably selected, and examples thereof include polar solvents such as ketone solvents, ester solvents, alcohol solvents, amide solvents, and ether solvents; hydrocarbon solvents; and alkaline aqueous solutions. Examples of the alkaline compound contained in the alkaline aqueous solution include mono-, di-, or tri-alkylamines; mono-, di-, or tri-alkanolamines; heterocyclic amines; tetraalkylammoniumhydroxydes; choline; 1,8-diazabicyclo[5,4,0]-7-undecene, and 1,5-diazabicyclo[4,3,0]-5-nonene.

Examples of the development method include a method in which the substrate is immersed in a tank filled with a developer for a certain time (dipping method), a method in which a developer is allowed to be swelled on the substrate surface by the surface tension and to stand still for a certain time for development (puddling method), a method in which a developer is sprayed on the substrate surface (spraying method), and a method in which a developer is continuously discharged on a substrate rotating at a certain speed while scanning a developer discharge nozzle at a certain speed (dynamic dispensing method).

The development time is not particularly limited, and is preferably 10 seconds to 90 seconds.

After development, a step of stopping development by replacing the developer with other solvents may be conducted.

Then, after development, a step of cleaning the resist film with a rinse liquid containing an organic solvent is preferably carried out.

The rinse liquid used in the rinsing step after development is not particularly limited, as long as the formed resist pattern is not dissolved, and a typical solution containing an organic solvent or water can be used.

As the rinse liquid, it is preferable to use a rinse liquid containing at least one organic solvent selected from a hydrocarbon solvent, a ketone solvent, an ester solvent, an alcohol solvent, an amide solvent, and an ether solvent.

The time for performing the rinsing step is not particularly limited, and is preferably 10 seconds to 90 seconds.

In the rinsing step, the developed substrate is subjected to cleaning treatment with the rinse liquid containing an organic solvent. Examples of the method for the cleaning treatment include, but are not particularly limited to, a method in which a rinse liquid is continuously discharged on a substrate rotating at a certain speed (spin coating method), a method in which the substrate is immersed in a tank filled with a rinse liquid for a certain time (dipping method), and a method in which a rinse liquid is sprayed on the substrate surface (spraying method).

A pattern wiring board can be obtained by forming the resist pattern and then performing etching. Etching can be performed by a known method such as dry etching using plasma gas, and wet etching with an alkaline solution, a cupric chloride solution, a ferric chloride solution, or the like.

After the resist pattern is formed, plating may be performed.

Examples of the plating method include, but are not particularly limited to, copper plating, solder plating, nickel plating, and gold plating.

The remaining resist pattern after etching can be stripped by an organic solvent.

Examples of the organic solvent include, but are not particularly limited to, PGMEA (propylene glycol monomethyl ether acetate), PGME (propylene glycol monomethyl ether), and EL (ethyl lactate). Examples of the above stripping method include, but are not particularly limited to, an immersion method and a spraying method. The wiring board on which the resist pattern is formed may be a multilayer wiring board, or may have a small diameter through hole.

In the present embodiment, the wiring board may be formed after resist pattern formation by a method in which a metal is vapor deposited in vacuum, followed by dissolving the resist pattern by a solution, that is, a lift-off method.

### EXAMPLES

Hereinafter, the present invention will be described by way of Examples, but the present invention is not limited by these Examples in any way. The measurement values in Examples were found by using the following method or apparatus.

### (1) Film Thickness of Coating Film

The film thickness of a coating film formed from the resist composition was measured using a film thicknesses measurement system (apparatus name “F20”, manufactured by Filmetrics, Inc.) in a constant-temperature constant-humidity chamber with a temperature of 23° C. and a humidity of 50% (relative humidity).

### (2) Content of Constitutional Unit of Resin

The content of the constitutional unit of a resin was measured by performing 1024 scans in the quantitative mode of ¹³C using ¹³C-NMR (model name “JNM-ECA500”, manufactured by JEOL Ltd., 125 MHz) with chloroform-d as a solvent.

### (3) Weight Average Molecular Weight (Mw), Number Average Molecular Weight (Mn), and Molecular Weight Distribution (Mw/Mn) of Resin

Mw and Mn of the resin were measured, in terms of polystyrene as a standard, by gel permeation chromatography (GPC) under the following conditions.
Apparatus name: LaChrom series manufactured by Hitachi, Ltd.
Detector: RI detector L-2490
Column: two TSKgel GMHHR-M columns+guard column HHR-H manufactured by Tosoh Corporation
Solvent: THF (with a stabilizer)
Flow rate: 1 mL/min
Column temperature: 40° C.

Then, the ratio of the calculated Mw to Mn [Mw/Mn] of the resin was calculated as the value of the molecular weight distribution of the resin.

The solvents used in the following Examples and Comparative Examples were as follows.

### <Component (B1)>

HBM: methyl 2-hydroxyisobutyrate, a compound in which R¹ is a methyl group in the general formula (b-1).

iPHIB: isopropyl 2-hydroxyisobutyrate, a compound in which R¹ is an i-propyl group in the general formula (b-1).

iBHIB: isobutyl 2-hydroxyisobutyrate, a compound in which R¹ is an i-butyl group in the general formula (b-1).

nBHIB: n-butyl 2-hydroxyisobutyrate, a compound in which R¹ is an n-butyl group in the general formula (b-1).

### <Component (B2)>

PGMEA: propylene glycol monomethyl ether acetate
MMP: methyl 3-methoxypropionate
nBuOAc: n-butyl acetate
EL: ethyl lactate

### [Resist Composition Containing Liquid Crystal Resin]

### Examples 1a to 47a, Comparative Examples 1a to 6a

A cresol novolac resin obtained by mixing “EP4080G” and “EP4050G” (both manufactured by ASAHI YUKIZAI CORPORATION) in the ratio of 1:1 (mass ratio) was used as a liquid crystal resin.

84 Parts by mass of the above cresol novolac resin and 16 parts by mass of a diazonaphthoquinone photosensitizer (trade name “DTEP-350” manufactured by DAITO CHEMIX Co., Ltd.) were mixed into a solvent of the kind and the blending ratio described in Table 1 and dissolved therein to prepare a resist composition having an active component (the above cresol novolac resin and photosensitizer) concentration described in Table 1 and Table 2.

Then, a coating film was formed from the prepared resist composition on a silicon wafer by spin coating at 1600 rpm, and the coating film was subjected to prebaking at 110° C. for 90 seconds to form a resist film. The film thicknesses were measured at randomly selected 5 points on the resist film, and the average value of the film thicknesses at 5 points was calculated as the average film thickness. The results are shown in Table 1 and Table 2.

TABLE 1

| | Kind and amount of | | | |
|---|---|---|---|---|
| | solvent blended | | | |
| | | Amount | Active | |
| | | blended | component | Film |
| | | (% by | concentration | thickness |
| | Kind | mass) | (% by mass) | (nm) |
| Example la | HBM | 100 | 25 | 2920 |
| Example 2a | | | 20 | 1680 |
| Example 3a | | | 18 | 1350 |
| Example 4a | | | 16 | 1050 |
| Example 5a | iPHIB | 100 | 22 | 2100 |
| Example 6a | | | 20 | 1550 |
| Example 7a | | | 18 | 1200 |
| Example 8a | iBHIB | 100 | 25 | 2850 |
| Example 9a | | | 22 | 1850 |
| Example 10a | | | 20 | 1450 |
| Example 11a | | | 18 | 1030 |
| Example 12a | nBHIB | 100 | 25 | 2450 |
| Example 13a | | | 22 | 1550 |
| Example 14a | | | 20 | 1200 |
| Comparative | PGMEA | 100 | 25 | 1850 |
| Example la | | | | |
| Comparative | | | 23 | 1480 |
| Example 2a | | | | |
| Comparative | | | 21 | 1180 |
| Example 3a | | | | |
| Comparative | EL | 100 | 25 | 2050 |
| Example 4a | | | | |
| Comparative | | | 23 | 1650 |
| Example 5a | | | | |
| Comparative | | | 21 | 1300 |
| Example 6a | | | | |

TABLE 2

| | Kind and amount of solvent blended | | | | | |
|---|---|---|---|---|---|---|
| | | Amount | | Amount | | |
| | | blended | | blended | Active component | |
| | | (% by | | (% by | concentration (% | Average film |
| | Kind | mass) | Kind | mass) | by mass) | thickness (nm) |
| Example 15a | HBM | 70 | PGMEA | 30 | 25 | 2560 |
| Example 16a | | | | | 22 | 1850 |
| Example 17a | | | | | 20 | 1460 |
| Example 18a | HBM | 30 | PGMEA | 70 | 25 | 2090 |
| Example 19a | | | | | 22 | 1550 |
| Example 20a | | | | | 20 | 1250 |
| Example 21a | HBM | 70 | MMP | 30 | 25 | 2390 |
| Example 22a | | | | | 22 | 1680 |
| Example 23a | | | | | 20 | 1390 |
| Example 24a | HBM | 30 | MMP | 70 | 25 | 1870 |
| Example 25a | | | | | 22 | 1360 |
| Example 26a | | | | | 20 | 1110 |
| Example 27a | HBM | 80 | nBuOAc | 20 | 25 | 2890 |
| Example 28a | | | | | 22 | 2040 |
| Example 29a | | | | | 20 | 1650 |
| Example 30a | iPHIB | 70 | PGMEA | 30 | 25 | 2450 |
| Example 31a | | | | | 22 | 1710 |
| Example 32a | | | | | 20 | 1350 |
| Example 33a | iPHIB | 30 | PGMEA | 70 | 25 | 2080 |
| Example 34a | | | | | 22 | 1490 |
| Example 35a | | | | | 20 | 1190 |
| Example 36a | iBHIB | 70 | PGMEA | 30 | 25 | 2670 |
| Example 37a | | | | | 22 | 1780 |
| Example 38a | | | | | 20 | 1280 |
| Example 39a | iBHIB | 30 | PGMEA | 70 | 25 | 2060 |
| Example 40a | | | | | 22 | 1460 |
| Example 41a | | | | | 20 | 1160 |
| Example 42a | nBHIB | 70 | PGMEA | 30 | 25 | 2560 |
| Example 43a | | | | | 22 | 1600 |
| Example 44a | | | | | 20 | 1250 |
| Example 45a | nBHIB | 30 | PGMEA | 70 | 25 | 2010 |
| Example 46a | | | | | 22 | 1360 |
| Example 47a | | | | | 20 | 1130 |

It is found from Table 1 that the resist compositions prepared in Examples 1a to 14a can form thick resist films as compared with the resist compositions of Comparative Examples 1a to 6a each having a comparable resin concentration.

It is also found from Table 2 that the resist compositions prepared in Examples 15a to 47a can form thick resist films, although the content of the liquid crystal resin is as low as 20 to 25% by mass.

### [Resist Composition Containing Resin for KrF]

### Examples 1b to 35b, Comparative Examples 1b to 19b

A copolymer having a constitutional unit of hydroxystyrene/t-butyl acrylate=2/1 (molar ratio) (manufactured by Maruzen Petrochemical Co., Ltd., Mw=20,000) was used as the resin for KrF.

The above copolymer and a mixed solvent of the kind and the blending ratio shown in Table 3 and Table 4 were mixed to prepare a resist composition having an active component (resin for KrF) concentration described in Table 3 and Table 4.

Then, a coating film was formed from the prepared resist composition on a silicon wafer by spin coating at 1600 rpm, and the coating film was subjected to prebaking at 110° C. for 90 seconds to form a resist film. The film thicknesses were measured at randomly selected 5 points on the resist film, and the average value of the film thicknesses at 5 points was calculated as the average film thickness. The results are shown in Table 3 and Table 4.

TABLE 3

| | | Active component | |
|---|---|---|---|
| | Kind of | concentration (% | Average film |
| | solvent | by mass) | thickness (nm) |
| Example 1b | HBM | 35 | 25700 |
| Example 2b | | 30 | 10600 |
| Example 3b | | 20 | 2430 |
| Example 4b | | 15 | 1030 |
| Example 5b | | 10 | 547 |
| Example 6b | iPHIB | 35 | 26000 |
| Example 7b | | 30 | 11000 |
| Example 8b | | 20 | 2110 |
| Example 9b | | 15 | 834 |
| Example 10b | | 10 | 436 |
| Example 11b | iBHIB | 35 | 35700 |
| Example 12b | | 30 | 15200 |
| Example 13b | | 20 | 2320 |
| Example 14b | | 15 | 811 |
| Example 15b | | 10 | 418 |
| Example 16b | nBHIB | 35 | 26900 |
| Example 17b | | 30 | 12300 |
| Example 18b | | 20 | 2100 |
| Example 19b | | 15 | 786 |
| Example 20b | | 10 | 400 |
| Example 27b | Mixed solvent of | 35 | 18200 |
| Example 28b | HBM/nBuOAc = | 30 | 8470 |
| Example 29b | 8/2 (mass ratio) | 20 | 2350 |
| Example 30b | | 10 | 716 |
| Example 31b | Mixed solvent of | 40 | 25000 |
| Example 32b | HBM/MMP = 5/5 | 35 | 13500 |
| Example 33b | (mass ratio) | 30 | 5680 |
| Example 34b | | 20 | 2170 |
| Example 35b | | 10 | 562 |

TABLE 4

| | | Active component | |
|---|---|---|---|
| | | concentration (% | Average film |
| | Kind of solvent | by mass) | thickness (nm) |
| Comparative | PGMEA | 40 | 21700 |
| Example 1b | | | |
| Comparative | | 35 | 11200 |
| Example 2b | | | |
| Comparative | | 30 | 5050 |
| Example 3b | | | |
| Comparative | | 20 | 1430 |
| Example 4b | | | |
| Comparative | | 15 | 555 |
| Example 5b | | | |
| Comparative | | 10 | 444 |
| Example 6b | | | |
| Comparative | MMP | 30 | 4190 |
| Example 7b | | | |
| Comparative | | 20 | 1350 |
| Example 8b | | | |
| Comparative | | 15 | 676 |
| Example 9b | | | |
| Comparative | | 10 | 378 |
| Example 10b | | | |
| Comparative | nBuOAc | 30 | 7380 |
| Example 11b | | | |
| Comparative | | 20 | 2070 |
| Example 12b | | | |
| Comparative | | 15 | 1010 |
| Example 13b | | | |
| Comparative | | 10 | 481 |
| Example 14b | | | |
| Comparative | EL | 35 | 15300 |
| Example 15b | | | |
| Comparative | | 30 | 7700 |
| Example 16b | | | |
| Comparative | | 20 | 1870 |
| Example 17b | | | |
| Comparative | | 15 | 777 |
| Example 18b | | | |
| Comparative | | 10 | 429 |
| Example 19b | | | |

It is found from Table 3 and Table 4 that the resist compositions prepared in Examples 1b to 35b can form thick resist films as compared with the resist compositions of Comparative Examples 1b to 19b having the same resin concentration.

### [Resist Composition Containing ArF Resins]

### Synthetic Examples 1 to 6 (Synthesis of ArF Resins (i) to (vi))

### (1) Monomer as Starting Material

In the synthesis of the ArF resins (i) to (vi), the following monomers as starting materials were used. The structure of each monomer as the starting material is as shown in Table 5.
EADM: 2-ethyl-2-adamantyl methacrylate
MADM: 2-methyl-2-adamantyl methacrylate
NML: 2-methacryloxy-4-oxatricyclo[4.2.1.0^{3.7}]nonan-5-one
GBLM: α-methacryloxy-y-butyrolactone
HADM: 3-hydroxy-1-adamantyl methacrylate

TABLE 5

| Monomer as | | | | | |
|---|---|---|---|---|---|
| starting | | | | | |
| material | EADM | MADM | NML | GBLM | HADM |
| Compound | 2-Ethyl-2-adamantyl | 2-Methyl-2-adamantyl | 2-Methacloyloxy-4- | α-Methacloyloxy- | 3-Hydroxy-1- |
| name | methacrylate | methacrylate | oxatricyclo[4.2.1.0^{3.7}]- | γ-butyrolactone | adamantyl |
| | | | nonan-5-one | | methacrylate |
| Structure | 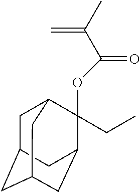 [SMILES: C=C(C)C(=O)OC1(CC)C2CC3CC(C2)CC1C3] | 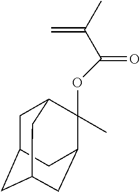 [SMILES: C=C(C)C(=O)OC1(C)C2CC3CC(C2)CC1C3] | 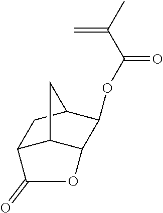 [SMILES: C=C(C)C(=O)OC1C2CC3C(=O)OC1C3C2] | 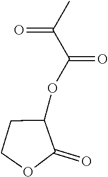 | 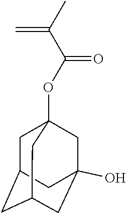 [SMILES: C=C(C)C(=O)OC12CC3CC(CC(O)(C3)C1)C2] |

### (2) Synthesis of ArF Resins (i) to (vi)

In a 300 mL round bottomed flask, 10 g in total of the monomers as starting materials were blended according to the kind and the molar ratio described in Table 6, and 300 g of tetrahydrofuran (manufactured by Wako Pure Chemical Industries, Ltd., guaranteed reagent, free from stabilizer) was further added, followed by stirring. Then, the mixture was degassed for 30 minutes in a nitrogen stream. After degassing, 0.95 g of 2,2′-azobis(isobutyronitrile) (manufactured by Tokyo Kasei Kogyo Co., Ltd., reagent) was added, and a polymerization reaction was conducted at 60° C. in a nitrogen stream so as to obtain a resin having a desired molecular weight.

After the completion of the reaction, the reaction solution cooled to room temperature (25° C.) was added dropwise to a large excess amount of hexane to precipitate a polymerization product. The precipitated polymerization product was collected by filtering, and the obtained solid was washed with methanol and then dried under reduced pressure at 50° C. for 24 hours to obtain each of the intended ArF resins (i) to (vi).

The content of each constitutional unit, and the Mw, Mn, and Mw/Mn of the obtained ArF resins (i) to (vi) were measured and calculated based on the aforementioned measurement method. The results are shown in Table 6.

TABLE 6

| | Formulation of monomers as starting materials (molar | | | | | | | | | | | | | |
|---|---|---|---|---|---|---|---|---|---|---|---|---|---|---|
| | ratio) | | | | | Content of constitutional unit of resin (mol %) | | | | | | Molecular weight of resin | | |
| ArF resin | EADM | MADM | NML | GBLM | HADM | EADM | MADM | NML | GBLM | HADM | Total | Mw | Mn | Mw/Mn |
| Resin (i) | 40 | | 40 | | 20 | 32.8 | | 38.3 | | 28.9 | 100 | 5940 | 3260 | 1.82 |
| Resin (ii) | 40 | | | 40 | 20 | 30.7 | | | 43.9 | 25.4 | 100 | 9850 | 4820 | 2.04 |
| Resin (iii) | | 40 | 40 | | 20 | | 38.0 | 37.7 | | 24.3 | 100 | 6480 | 3160 | 2.05 |
| Resin (iv) | | 40 | | 40 | 20 | | 23.7 | | 44.9 | 31.4 | 100 | 15800 | 6820 | 2.32 |
| Resin (v) | | 40 | 40 | | 20 | | 41.6 | 33.0 | | 25.4 | 100 | 14200 | 7260 | 1.96 |
| Resin (vi) | | 40 | | 60 | | | 25.4 | | 74.6 | | 100 | 11500 | 3770 | 3.05 |

### Examples 1c to 18c, Comparative Examples 1c to 12c

Any of the resins for ArF (i) to (vi) obtained in the above Synthetic Examples 1 to 6 was mixed with a solvent of the kind shown in Tables 7 and 8 to prepare a resist composition having an active component (resins for ArF) concentration described in Tables 7 and 8.

Then, a coating film was formed from the prepared resist composition on a silicon wafer by spin coating at 3000 rpm, and the coating film was subjected to prebaking at 90° C. for 60 seconds to form a resist film. The film thicknesses were measured at randomly selected 5 points on the resist film, and the average value of the film thicknesses at 5 points was calculated as the average film thickness. The results are shown in Table 7 and Table 8.

TABLE 7

| | | | Active component | Average |
|---|---|---|---|---|
| | | Kind of | concentration | film thick- |
| | ArF resin | solvent | (% by mass) | ness (nm) |
| Example 1c | Resin (i) | HBM | 10 | 343 |
| Example 2c | | | 5 | 131 |
| Example 3c | | | 2 | 45.7 |
| Example 4c | Resin (ii) | HBM | 10 | 373 |
| Example 5c | | | 5 | 132 |
| Example 6c | | | 2 | 42.1 |
| Example 7c | Resin (iii) | HBM | 10 | 361 |
| Example 8c | | | 5 | 142 |
| Example 9c | | | 2 | 44.9 |
| Example 10c | Resin (iv) | HBM | 10 | 401 |
| Example 11c | | | 5 | 142 |
| Example 12c | | | 2 | 44.7 |
| Example 13c | Resin (v) | HBM | 10 | 420 |
| Example 14c | | | 5 | 143 |
| Example 15c | | | 2 | 44.1 |
| Example 16c | | | 10 | 343 |
| Example 17c | Resin (vi) | HBM | 5 | 133 |
| Example 18c | | | 2 | 45.4 |

TABLE 8

| | | | Active component | Average |
|---|---|---|---|---|
| | | Kind of | concentration | film thick- |
| | ArF resin | solvent | (% by mass) | ness (nm) |
| Comparative | Resin (i) | PGMEA | 10 | 215 |
| Example 1c | | | | |
| Comparative | | | 5 | 95.7 |
| Example 2c | | | | |
| Comparative | | | 2 | 28.9 |
| Example 3c | | | | |
| Comparative | Resin (ii) | PGMEA | 10 | 237 |
| Example 4c | | | | |
| Comparative | | | 5 | 90.0 |
| Example 5c | | | | |
| Comparative | | | 2 | 28.1 |
| Example 6c | | | | |
| Comparative | Resin (iii) | PGMEA | 10 | 232 |
| Example 7c | | | | |
| Comparative | | | 5 | 92.3 |
| Example 8c | | | | |
| Comparative | | | 2 | 33.9 |
| Example 9c | | | | |
| Comparative | Resin (iv) | PGMEA | 10 | 256 |
| Example 10c | | | | |
| Comparative | | | 5 | 97.2 |
| Example 11c | | | | |
| Comparative | | | 2 | 29.8 |
| Example 12c | | | | |

It is found from Table 7 and Table 8 that the resist compositions prepared in Examples 1c to 18c can form thick resist films as compared with the resist compositions of Comparative Examples 1c to 12c having the same resin concentration.

### Example 1d, Comparative Example 1d

### <Resist Performance>

The results of the following evaluation of the resin (ii) for resist performance are shown in Table 9.

### (Preparation of Resist Composition)

Resist compositions were prepared according to the formulation shown in Table 9. The followings were used as the acid generating agent (C) and the solvent in components of the resist compositions shown in Table 9.

### Acid Generating Agent (C)

P-1: triphenylsulfonium trifluoro-1-butanesulfonate (Sigma-Aldrich) Solvent
S-1: methyl 2-hydroxyisobutyrate (manufactured by Mitsubishi Gas Chemical Company, Inc.)
S-1: propylene glycol monomethyl ether acetate (manufactured by Kanto Chemical Co., Inc.)

TABLE 9

| | Resist compositions | | | | | |
|---|---|---|---|---|---|---|
| | Resin (ii) | P-1 | S-1 | S-2 | Evaluation results | |
| | [parts by | [parts by | [parts by | [parts by | Pattern | Pattern |
| | mass] | mass] | mass] | mass] | evaluation | film thickness |
| Example 1d | 2.3 | 0.2 | 97.5 | — | Good | Good |
| Comparative | 2.7 | 0.2 | — | 97.1 | Good | Poor |
| Example 1d | | | | | | |

### (Evaluation Method of Resist Performance of Resist Composition)

The resist composition was uniformly applied to a clean silicon wafer by spin coating, which was then pre-baked (PB) before exposure using a hot plate at 90° C. to form a resist film having a thickness of 50 nm. The obtained resist film was irradiated with an electron beam set to 1:1 line and space with a pitch of 500 nm using an electron beam lithography apparatus (ELS-7500, manufactured by ELIONIX INC.). After irradiation, the resist film was heated at 90° C. for 90 seconds and immersed in an alkaline developer containing 2.38% by mass of tetramethylammonium hydroxide (TMAH) for 60 seconds for development. Thereafter, the resist film was washed with ultrapure water for 30 seconds and dried to form a resist pattern. On the formed resist pattern, the line and space were observed with a scanning electron microscope (S-4800 manufactured by Hitachi High-Technologies Corporation), and the reactivity of the resist composition by irradiation with the electron beam was evaluated.

As for the resist pattern evaluation, a good resist pattern was obtained in both Example 1d and Comparative Example 1d by irradiating the resist film with an electron beam set to 1:1 line and space with a pitch of 500 nm. In addition, regarding the film thickness of the resist pattern, the film thickness of Example 1d was thick and demonstrated to have sufficient etching resistance to transfer the resist pattern. On the other hand, the film thickness of Comparative Example 1d was thin and demonstrated to have no etching resistance required for pattern transfer.

Thus, when the resist composition that satisfies the requirement of the present embodiment is used, a good resist pattern shape can be obtained as compared with the resist composition of Comparative Example 1d, which does not satisfy the requirement. Also, resist compositions other than those described in Examples exhibit the same effect, as long as the above requirement of the present embodiment is satisfied.

### [Resist Composition Containing Resin for Arf Resist and Acid Generating Agent]

Resist compositions were prepared according to the formulation shown in Table 10 and Table 11, and the evaluation of the solubility of the resins for ArF (i) to (v) and the acid generating agents (i) to (iv), which were used as materials, shown in Table 10 and Table 11 was performed.

### <Solvent>

HBM: methyl 2-hydroxyisobutyrate (manufactured by Mitsubishi Gas Chemical Company, Inc.)
αMBM: methyl α-methoxyisobutyrate (synthesized with reference to “US2014/0275016”)
αFBM: methyl α-formyloxyisobutyrate (synthesized with reference to “WO2020/004467”)
αABM: methyl α-acetyloxyisobutyrate (synthesized with reference to “WO2020/004466”)
3HBM: methyl 3-hydroxyisobutyrate (manufactured by Tokyo Kasei Kogyo Co., Ltd.)
PGME: 1-methoxy-2-propanol (manufactured by Sigma-Aldrich)

### <Resin>

The resins having the following compositional ratios (molecular weight) were synthesized by the above methods.
(i) EADM/NML=18/82 (Mn=3750)
(ii) MADM/NML=25/75 (Mn=2740)
(iii) MADM/GBLM=25/75 (Mn=3770)
(iv) MADM/NML/HADM=42/33/25 (Mn=7260)
(v) a copolymer having constitutional units of hydroxystyrene/t-butyl acrylate/styrene=3/1/1 (molar ratio) (manufactured by Maruzen Petrochemical Co., Ltd., Mw=12,000)

### <Acid Generating Agent>

(i) WPAG-336 (manufactured by FUJIFILM Wako Pure Chemical Corporation)
(ii) WPAG-367 (manufactured by FUJIFILM Wako Pure Chemical Corporation)
(iii) WPAG-145 (manufactured by FUJIFILM Wako Pure Chemical Corporation)

### (iv) Triphenylsulfonium Trifluoro-1-Butanesulfonate (Sigma-Aldrich)

Each resin of the kind shown in Table 10 was added to each solvent of the kind shown in Table 10 such that the resin concentration was 15 wt %, and each acid generating agent of the kind shown in Table 10 was added to the mixture such that the acid generating agent concentration was 1 wt %, to prepare each of the resist compositions of Examples A1-1 to A1-4 and Comparative Example A1-1. The state after stirring at room temperature for 24 hours was visually evaluated according to the following criteria.
Rank S: Dissolved (clear solution was visually found)
Rank A: Almost dissolved (almost clear solution was visually found)
Rank C: Insoluble (cloudy solution was visually found)

Each resin shown in Table 11 was added to each solvent shown in Table 11 such that the resin concentration was 40 wt %, and each acid generating agent of the kind shown in Table 11 was added to the mixture such that the acid generating agent concentration was a predetermined concentration, to prepare each of the resist compositions of Examples A2-1a to A2-5d and Comparative Example A2-1. The state after stirring at room temperature for 1 hour was visually evaluated according to the following criteria.
Rank S: 5 wt % Dissolved (clear solution was visually found)
Rank A: 1 wt % Dissolved (clear solution was visually found)
Rank C: 1 wt % Insoluble (cloudy solution was visually found)

The results are shown in Table 10 and Table 11.

TABLE 10

| | | Resin | Resin | Resin | Resin |
|---|---|---|---|---|---|
| | | (i) | (ii) | (iii) | (iv) |
| | | Acid | Acid | Acid | Acid |
| | | gener- | gener- | gener- | gener- |
| | | ating | ating | ating | ating |
| | | agent | agent | agent | agent |
| | Solvent | (iv) | (iv) | (iv) | (iv) |
| Example | HBM/αMBM | A | A | S | S |
| A1-1 | (1:1 weight | | | | |
| | ratio) | | | | |
| Example | HBM/αFBM | S | S | S | S |
| A1-2 | (1:1 weight | | | | |
| | ratio) | | | | |
| Example | HBM/3HBM | A | A | S | S |
| A1-3 | (1:1 weight | | | | |
| | ratio) | | | | |
| Example | HBM | A | A | S | S |
| A1-4 | | | | | |
| Example | HBM/PGME | A | A | S | S |
| A1-5 | (1:1 weight | | | | |
| | ratio) | | | | |
| Compara- | PGMEA | C | C | C | C |
| tive | | | | | |
| Example | | | | | |
| A1-1 | | | | | |

TABLE 11-1

| | | Resin | Resin | Resin |
|---|---|---|---|---|
| | | (v) | (v) | (v) |
| | | Acid | Acid | Acid |
| | | generating | generating | generating |
| | | agent | agent | agent |
| | Solvent | (i) | (ii) | (iii) |
| Example | HBM/αMBM | S | S | S |
| A2-1a | (1:1 weight | | | |
| | ratio) | | | |
| Example | HBM/αMBM | S | S | S |
| A2-1b | (9:1 weight | | | |
| | ratio) | | | |
| Example | HBM/αMBM | S | S | S |
| A2-1c | (1:0.001 weight | | | |
| | ratio) | | | |
| Example | HBM/αMBM | S | S | S |
| A2-1d | (1:0.00005 weight | | | |
| | ratio) | | | |
| Example | HBM/αFBM | S | S | S |
| A2-2a | (1:1 weight | | | |
| | ratio) | | | |

TABLE 11-2

| | | | | |
|---|---|---|---|---|
| Example A2-2b | HBM/αFBM | S | S | S |
| | (9:1 weight ratio) | | | |
| Example A2-2c | HBM/αFBM | S | S | S |
| | (1:0.001 weight ratio) | | | |
| Example A2-2d | HBM/αFBM | S | S | S |
| | (1:0.00005 weight ratio) | | | |
| Example A2-3a | HBM/3HBM | S | S | S |
| | (1:1 weight ratio) | | | |
| Example A2-3b | HBM/3HBM | S | S | S |
| | (9:1 weight ratio) | | | |
| Example A2-3c | HBM/3HBM | S | S | S |
| | (1:0.001 weight ratio) | | | |
| Example A2-3d | HBM/3HBM | S | S | S |
| | (1:0.00005 weight ratio) | | | |
| Example A2-4 | HBM | S | S | A |
| Example A2-5a | HBM/PGME | S | S | S |
| | (1:1 weight ratio) | | | |
| Example A2-5b | HBM/PGME | S | S | S |
| | (9:1 weight ratio) | | | |
| Example A2-5c | HBM/PGME | S | S | S |
| | (1:0.001 weight ratio) | | | |
| Example A2-5d | HBM/PGME | S | S | S |
| | (1:0.00005 weight ratio) | | | |
| Comparative | PGMEA | A | C | A |
| Example A2-1 | | | | |

It is found from Table 10 that the resist compositions prepared in Examples A1-1 to A1-5 are excellent in the solubility of the resin and can prepare various resist compositions as compared with the resist composition of Comparative Example A1-1. In particular, the resist compositions in which the solvent (B) contains αFBM as the solvent (B2) exhibit high solubility of any of the resins and are suitably used.

It is found from Table 12 that the resist auxiliary film compositions prepared in Examples A2-1a to A2-5d are excellent in the solubility of any acid generating agents as compared with the resist composition of Comparative Example A2-1, and that the resist composition can be thus prepared even when any of the acid generating agents is used. In particular, the resist compositions in which the solvent (B) contains αMBM, αFBM, or 3HBM as the solvent (B2) exhibit high solubility of any acid generating agents and are suitably used.

### [Resist Composition Containing Resin for KrF]

A copolymer having constitutional units of hydroxystyrene/t-butyl acrylate/styrene=3/1/1 (molar ratio) as the resin for KrF (manufactured by Maruzen Petrochemical Co., Ltd., Mw=12,000) was mixed with the solvent of the kind shown in Table 12 to prepare a resist composition having an active component (resin for KrF) concentration described in Table 12.

Then, a coating film was formed from the prepared resist composition on a silicon wafer by spin coating at 1500 rpm, and the coating film was subjected to prebaking at 140° C. for 60 seconds to form a resist film. The film thicknesses were measured at randomly selected 5 points on the resist film, and the average value of the film thicknesses at 5 points was calculated as the average film thickness, and the film thickness was evaluated. In addition, the difference between the maximum value and the minimum value of the film thicknesses was divided by the average value, and the result was evaluated as film uniformity. The results are shown in Table 12.

### Film Thicknesses:

Rank A: 20 μm or more
Rank B: 15 μm or more and less than 20 μm
Rank C: less than 15 μm

### Film Uniformity:

Rank A: less than 15
Rank B: 15 or more and less than 30
Rank C: 30 or more

TABLE 12

| | | Thick- | film | Film |
|---|---|---|---|---|
| | | ness | thick- | unifor- |
| | | wt % | ness | mity |
| Example A3-1a | HBM/αMBM | 40 | B | A |
| | (1:1 weight ratio) | | | |
| Example A3-1b | HBM/αMBM | 45 | A | A |
| | (1:1 weight ratio) | | | |
| Example A3-1c | HBM/αMBM | 45 | A | A |
| | (1:0.001 weight ratio) | | | |
| Example A3-2a | HBM/αFBM | 40 | A | A |
| | (1:1 weight ratio) | | | |
| Example A3-2b | HBM/αFBM | 40 | A | B |
| | (1:2 weight ratio) | | | |
| Example A3-2c | HBM/αFBM | 40 | A | A |
| | (1:0.001 weight ratio) | | | |
| Example A3-3a | HBM/3HBM | 40 | B | A |
| | (1:1 weight ratio) | | | |
| Example A3-3b | HBM/3HBM | 40 | B | A |
| | (9:1 weight ratio) | | | |
| Example A3-3c | HBM/3HBM | 40 | B | A |
| | (1:0.001 weight ratio) | | | |
| Example A3-4a | HBM | 40 | B | A |
| Example A3-4b | HBM | 45 | B | B |
| Example A3-5a | HBM/PGME | 40 | B | A |
| | (1:1 weight ratio) | | | |
| Example A3-5b | HBM/PGME | 45 | B | A |
| | (9:1 weight ratio) | | | |
| Example A3-5c | HBM/PGME | 45 | B | A |
| | (1:0.001 weight ratio) | | | |
| Comparative Example | PGMEA | 40 | C | — |
| A3-1a | | | | |
| Comparative Example | PGMEA | 45 | C | — |
| A3-1b | | | | |

It is found from Table 12 that the resist compositions prepared in Examples A3-1a to A3-5c can form thick resist films as compared with the resist compositions of Comparative Examples A3-1a to A3-1b. In particular, the resist compositions in which the solvent (B) contains αMBM, αFBM, 3HBM, or PGME as the solvent (B2) are excellent in film uniformity and suitably used. In addition, the resist compositions containing &FBM can have a film thickness of 20 μm or more and are suitably used when the resin concentration is 40 wt %. Further, the resist compositions containing αMBM can have a resin concentration of 45 wt % and gives a film thickness of 20 μm or more, and such compositions are suitably used.

### <Evaluation of In-Plane Uniformity of Resist Film>

The resin for KrF (copolymer having constitutional units of hydroxystyrene/t-butyl acrylate/styrene=3/1/1 (molar ratio) (manufactured by Maruzen Petrochemical Co., Ltd., Mw=12,000)) was mixed with the solvent of the kind shown in Table 13 to prepare a resist composition having an active component (resin for KrF) concentration described in Table 13.

Then, a coating film was formed from the prepared resist composition on a silicon wafer at a main spin of 1200 rpm, and the coating film was subjected to prebaking at 110° C. for 90 seconds to form a resist film having an average film thickness of 7.2 μm. Film thicknesses were measured at 50 points positioned at a distance of 3 mm in the diameter direction on the resist film. A triple of the standard deviation of the film thickness was divided by the average film thickness to calculate the film thickness unevenness 30, and the in-plane uniformity was evaluated. The results are shown in Table 13.

### In-Plane Uniformity:

Rank A: 30≤ less than 0.02
Rank B: 0.02 or more and less than 0.04
Rank C: 0.04 or more

TABLE 13

| | | Concentration | In-plane |
|---|---|---|---|
| | | wt % | uniformity |
| Example A4-1a | HBM/PGME | 30.9 | A |
| | (3:7 weight ratio) | | |
| Example A4-1b | HBM/PGME | 30.1 | A |
| | (7:3 weight ratio) | | |
| Example A4-2a | HBM | 29.4 | B |
| Comparative | PGME | 31.8 | C |
| Example A4-1a | | | |

### <Resist Performance>

The results of the following evaluation of the resin (ii) (MADM/NML=25/75) for resist performance are shown in Table 14.

### Pattern Evaluation:

Rank S: A rectangular resist pattern is formed
Rank A: An almost rectangular resist pattern is formed
Rank C: No rectangular resist pattern is formed

### Pattern Film Thickness:

Rank A: having etching resistance required for pattern transfer
Rank C: having no etching resistance required for pattern transfer

### (Preparation of Resist Composition)

Resist compositions were prepared in according to the formulation shown in Table 14. The followings were used as the acid generating agent (C) and the solvent in components of the resist compositions shown in Table 14.

### Acid Generating Agent (C)

P-1: triphenylsulfonium trifluoro-1-butanesulfonate (Sigma-Aldrich)

TABLE 14

| | Resist compositions | | | | | |
|---|---|---|---|---|---|---|
| | Resin (ii) | P-1 | B1 | B2 | Evaluation results | |
| | [parts by | [parts by | [parts by | [parts by | Pattern | Pattern |
| | mass] | mass] | mass] | mass] | evaluation | film thickness |
| Example A5-1 | 2.3 | 0.2 | HBM | — | A | A |
| | | | 97.5 | | | |
| Example A5-2a | 2.3 | 0.2 | HBM | αMBM | A | A |
| | | | 48.8 | 48.8 | | |
| Example A5-2b | 2.3 | 0.2 | HBM | αMBM | A | A |
| | | | 97.5 | 0.098 | | |
| Example A5-3a | 2.3 | 0.2 | HBM | αFBM | A | A |
| | | | 48.8 | 48.8 | | |
| Example A5-3b | 2.3 | 0.2 | HBM | αFBM | A | A |
| | | | 97.5 | 0.098 | | |
| Example A5-4a | 2.3 | 0.2 | HBM | αABM | A | A |
| | | | 48.8 | 48.8 | | |
| Example A5-4b | 2.3 | 0.2 | HBM | αABM | A | A |
| | | | 97.5 | 0.098 | | |
| Example A5-5a | 2.3 | 0.2 | HBM | 3HBM | S | A |
| | | | 48.8 | 48.8 | | |
| Example A5-5b | 2.3 | 0.2 | HBM | 3HBM | S | A |
| | | | 97.5 | 0.098 | | |
| Example A5-6a | 2.3 | 0.2 | HBM | PGME | A | A |
| | | | 48.8 | 48.8 | | |
| Example A5-6b | 2.3 | 0.2 | HBM | PGME | A | A |
| | | | 97.5 | 0.098 | | |
| Comparative Example | 2.7 | 0.2 | — | PGMEA | A | C |
| A5 | | | | 97.1 | | |

### (Evaluation Method of Resist Performance of Resist Composition)

The resist composition was uniformly applied to a clean silicon wafer by spin coating, which was then pre-baked (PB) before exposure using a hot plate at 90° C. to form a resist film having a thickness of 50 nm. The obtained resist film was irradiated with an electron beam set to 1:1 line and space with a pitch of 500 nm using an electron beam lithography apparatus (ELS-7500, manufactured by ELIONIX INC.). After irradiation, the resist film was heated at 90° C. for 90 seconds and immersed in an alkaline developer containing 2.38% by mass of tetramethylammonium hydroxide (TMAH) for 60 seconds for development. Thereafter, the resist film was washed with ultrapure water for 30 seconds and dried to form a resist pattern. On the formed resist pattern, the line and space were observed with a scanning electron microscope (S-4800 manufactured by Hitachi High-Technologies Corporation), and the reactivity of the resist composition by irradiation with the electron beam was evaluated.

As for the resist pattern evaluation, a good resist pattern was obtained in Examples A5-1 to A5-6b and Comparative Example A5 by irradiating the resist film with an electron beam set to 1:1 line and space with a pitch of 500 nm. In addition, regarding the film thickness of the resist pattern, the film thickness of Examples A5-1 to A5-6b was thick and demonstrated to have sufficient etching resistance to transfer the resist pattern. On the other hand, the film thickness of Comparative Example A5 was thin and demonstrated to have no etching resistance required for pattern transfer. In particular, when using the resist compositions in which the solvent (B) contains 3HBM as the solvent (B2), the shape of the obtained resist pattern is a rectangle, and the resist compositions are excellent in pattern transfer performance and suitably used.

Thus, when the resist composition that satisfies the requirement of the present embodiment is used, a good resist pattern shape can be obtained as compared with the resist composition of Comparative Example A⁵, which does not satisfy the requirement.

Also, other resist compositions other than those described in Examples exhibit the same effect, as long as the above requirement of the present embodiment is satisfied.

1. A resist composition comprising:
(A) a resin, and
(B) a solvent comprising: (B1) a compound represented by the following general formula (b-1), wherein
a content of an active component is 45% by mass or less based on the total amount of the resist composition: 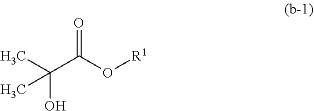
wherein R¹ is an alkyl group having 1 to 10 carbon atoms.

2. The resist composition according to claim 1, further comprising: (C) at least one additive selected from the group consisting of a photosensitizer and an acid generating agent.

3. The resist composition according to claim 1, wherein R¹ in the general formula (b-1) is a methyl group, an ethyl group, an n-propyl group, an i-propyl group, an n-butyl group, an i-butyl group, an s-butyl group, or a t-butyl group.

4. The resist composition according to claim 1, wherein R¹ in the general formula (b-1) is an ethyl group, an n-propyl group, an i-propyl group, an n-butyl group, an i-butyl group, an s-butyl group, or a t-butyl group.

5. The resist composition according to claim 1, wherein the solvent (B) comprises: (B2) a solvent other than the compound (B1).

6. The resist composition according to claim 5, wherein the solvent (B) comprises one or more selected from the group consisting of methyl α-methoxyisobutyrate, methyl α-formyloxyisobutyrate, methyl α-acetyloxyisobutyrate, and methyl 3-hydroxyisobutyrate, as the solvent (B2).

7. The resist composition according to claim 5, wherein the solvent (B) comprises one or more selected from the group consisting of methyl α-methoxyisobutyrate, methyl α-formyloxyisobutyrate, methyl α-acetyloxyisobutyrate, methyl 3-hydroxyisobutyrate, and 1-methoxy-2-propanol, as the solvent (B2).

8. The resist composition according to claim 5, wherein the solvent (B2) is contained in an amount of 100% by mass or less based on the total amount (100% by mass) of the compound (B1).

9. The resist composition according to claim 8, wherein the solvent (B2) is contained in an amount less than 70% by mass based on the total amount (100% by mass) of the compound (B1).

10. The resist composition according to claim 8, wherein the solvent (B2) is contained in an amount of 0.0001% by mass or more based on the total amount (100% by mass) of the compound (B1).

11. The resist composition according to claim 5, wherein the solvent (B2) is contained in an amount less than 100% by mass based on the total amount (100% by mass) of the resist composition.

12. The resist composition according to claim 1, wherein the resin (A) comprises a novolac resin (A¹).

13. The resist composition according to claim 1, wherein the resin (A) comprises: (A²) a resin having at least one of (a2-1) a constitutional unit derived from a phenolic hydroxyl group-containing compound, and (a2-2) a constitutional unit capable of being decomposed by an action of an acid, a base, or heat to form an acid functional group.

14. The resist composition according to claim 1, wherein the resin (A) comprises: (A³) a resin having (a3-1) a constitutional unit having an adamantane structure.

15. The resist composition according to claim 14, wherein the resin (A³) is a copolymer having (a3-2) a constitutional unit having a lactone structure together with the constitutional unit (a3-1).

16. The resist composition according to claim 14, wherein a content of (a3-1α) a constitutional unit having an adamantane structure substituted with a hydroxy group is less than 50 mol %, based on the total amount of the constitutional unit of the resin (A³).

17. The resist composition according to claim 1, wherein the resin (A) comprises: (A⁴) a resin having any two or more constitutional units of (a2-1) the constitutional unit derived from a phenolic hydroxyl group-containing compound, (a2-2) the constitutional unit capable of being decomposed by an action of an acid, a base, or heat to form an acid functional group, (a3-1) the constitutional unit having an adamantane structure, and (a3-2) the constitutional unit having a lactone structure.

18. A method for forming a resist film comprising:
applying the resist composition according to claim 1 on a substrate to form a coating film,
performing heat treatment after application of the resist composition on the substrate, and
forming a resist pattern.
